(19) United States
(12) Patent Application Publication
(10) Pub. No.: US 2023/0091493 A1
(43) Pub. Date: Mar. 23, 2023

(54) DNA SYNTHESIS YIELD IMPROVEMENTS

(71) Applicant: Touchlight IP Limited, London (GB)

(72) Inventors: Paul ROTHWELL, London (GB); Daniel KYSH, London (GB); Neil PORTER, London (GB)

(73) Assignees: Touchlight IP Limited, London (GB); Touchlight IP Limited, London (GB)

(21) Appl. No.: 17/799,353

(22) PCT Filed: Feb. 15, 2021
(86) PCT No.: PCT/GB2021/050366

§ 371 Date: Aug. 12, 2022

(30) Foreign Application Priority Data
Feb. 14, 2020 (GB) 2002068.1

(51) Int. Cl. C12P 19/34

(52) U.S. Cl. CPC C12P 19/34; C12Y 207/07007

(57) ABSTRACT

The present invention relates to an improved process for synthesis of deoxyribonucleic acid (DNA), in particular cell-free enzymatic synthesis of DNA, preferably on a large or industrial scale, with an improved yield and/or with an improved efficiency. The invention requires the use of nucleotide complexes wherein the nucleotide is associated with a mixture of divalent and monovalent cations. Preferably, the divalent cation may be magnesium or manganese.

### FIELD

The present invention relates to an improved process for synthesis of deoxyribonucleic acid (DNA), in particular cell-free enzymatic synthesis of DNA, preferably on a large or industrial scale, with an improved yield and/or with an improved efficiency.

### BACKGROUND

Amplification of deoxyribonucleic acid (DNA) may be carried out through use of cell-based processes, such as by culture of bacteria propagating DNA to be amplified in fermenters. Cell-free enzymatic processes for amplification of DNA from a starting template have also been described, including the polymerase chain reaction and strand-displacement reactions.

In the past, amplification of DNA on a test scale has been performed using apparatus based on microtitre plates and robotically controlled pipettes to add reaction components as required. Such apparatus and processes are suitable for manufacturing small quantities of DNA for test purposes but do not provide sufficient quantities for other purposes. Large scale amplification and manufacture of specific nucleic acids and proteins has mostly been carried out through cell-based processes. Such methods are generally effective for production of very large volumes of product but costly to set up. Further, it is preferable to synthesise DNA in a cell-free environment for clinical and therapeutic purposes. In terms of amplification of plasmid using routine methods in the art such as fermentation, a commercial scale operation may be able to manufacture 2.6 g/l. This is considered by those in the art to be at “industrial scale”.

Large-scale DNA synthesis using chemical synthesis, such as phosphoramidite methods, are known, but are not without drawbacks. The reaction must generally be performed in organic solvents, many of which are toxic or otherwise hazardous. Another drawback to chemical synthesis is that it is not completely efficient, since following each nucleotide addition, some percentage of the growing oligonucleotide chains are capped, resulting in a yield loss. The total yield loss for the nucleotide chain being synthesised thus increases with each nucleotide added to the sequence. This inherent inefficiency in chemical synthesis of oligonucleotides ultimately limits the length of oligonucleotide that can be efficiently produced to oligonucleotides having 50 nucleic acid residues or less, and furthermore affects the accuracy of synthesis.

To date, biological catalysts such as polymerase have not been routinely exploited for industrial scale manufacture of DNA products in vitro and reactions have largely been limited to volumes at microliter scale. Scaling up processes using enzymatic synthesis of DNA has proved problematic, not least with the disappointing yield of DNA product.

The present applicants have previously addressed the ability of scaling-up using commercially available nucleotides. A new process was developed that involved adding fresh nucleotides to the reaction mixture as they became depleted or as the concentration of the product reached a threshold, as described in WO2016/034849, incorporated herein by reference. However, it was established that even higher yields could be achieved and the inventors have developed a new method described herein to further enhance the yield from enzymatic DNA synthesis.

Enzymatic DNA synthesis generally requires the use of a polymerase or polymerase-like enzyme to catalyse the addition of nucleotides to a nascent nucleic acid chain. Generally, a template DNA is required which is amplified in the reaction. However, it is also possible to perform template-free DNA synthesis, where the incorporation happens de novo.

It is important to note that due to the highly charged nature of nucleic acids, they are constantly surrounded by counter-ions to neutralise most of their charges which lessen the electrostatic repulsion between sections of sequence, so they can be condensed into neat, compact structures in the cell. The building blocks of nucleic acids, the nucleotides, are also an ionic species and require the presence of positive counter-ions in order to maintain electrical neutrality. Most, if not all, nucleotides are thus supplied as salts with a positive counter-ion. Without a positive counter-ion from a salt, nucleotides are presented as their free acid form where electrical neutrality is maintained by a hydrogen ion. Since the nucleotide has four negative charges, salts are typically prepared with 2 divalent cations, or 4 monovalent cations. It will be apparent to those skilled in the art that as soon as a nucleotide (salt or acid) is dispersed in water or other solvent, they may dissociate in solution into anionic and cationic components.

In general, nucleotides are supplied for DNA synthesis, amplification or sequencing as either a lithium or sodium salt. Lithium is generally preferred since these salts offer greater solubility and also stability to repeated freezing and thawing cycles than sodium salts and remain sterile due to the bacteriostatic activity of lithium towards various microorganisms, giving greater reliability and an extended shelf life. The use of these salts is so routine that those skilled in the art do not appear to question the counter-ion present with the nucleotide. Indeed, all of the nucleotides used in the Examples of WO2016/034849 are lithium salts of nucleotides, since these are marketed as the superior choice to those skilled in the art. Nucleotides supplied as a salt only of divalent cations, such as magnesium ions, are highly desirable, since the same are required during enzymatic DNA synthesis as a co-factor. Unfortunately, nucleotides provided as magnesium salts are highly insoluble and this limits their use. Instead, magnesium is usually supplied to the reaction separately as a chloride salt, in combination with nucleotides that are counter-ioned with a monovalent cation species.

The present inventors have previously found that the species of cation present in the nucleotide salt as the counter-ion is critical to the yield, efficiency and fidelity of high yielding enzymatic DNA synthesis reaction, as detailed in PCT/GB2019/052307, herein incorporated by reference. This was rather unexpected, since commercially available nucleotides are available generally as only lithium or sodium salts. However, using alternative cations as counter-ions for the ionic nucleotides has a great impact on the DNA synthesis reaction, as can be seen from the Examples included in PCT/GB2019/052307, which uses various different monovalent cations to counter-ion the nucleotide, including potassium, ammonium and caesium. Further, the inventors have previously demonstrated that the specific counter-ion used in the nucleotide salt can alter the requirements of the DNA synthesis enzyme for magnesium in order to achieve a high yield of DNA.

The present inventors have now developed a way of pushing the maximum yield for a DNA synthesis reaction further still, by the use of a nucleotide which is effectively counter-ioned by a mixture of divalent and monovalent cations. In using a mixture of different entities to effectively counter-ion the nucleotide, the net effect is to reduce the amount of monovalent cation present. This is important, since it is postulated by the inventors that monovalent cations are inhibitory in large concentrations to the DNA synthesis. Moreover, by also providing a divalent cation as a counter-ion to the nucleotide, particularly in the case of magnesium or manganese, this can effectively also provide the co-factor required by the synthesis enzyme. Therefore, it is not necessary to provide further or additional divalent cation. Since magnesium or manganese are usually added to the reaction as a salt (including two negative charges on one or more anions), including them instead as a counter-ion for the nucleotide inherently reduces the amount of anion present. Thus, by providing a nucleotide associated with a mixture of monovalent and divalent counter-ions, many benefits are derived. Additionally, using a mixture of monovalent and divalent counter-ions has permitted the formulation of nucleotide complexes wherein less than 4 positive charges are supplied by the monovalent or divalent cations described herein. This is also beneficial, since it permits a further reduction in the amount of monovalent cations present in the reaction mixture, and further reduces the ionic strength of the reaction mixture. Such a reduction may be beneficial for downstream DNA processing enzymes, since at the end of the synthesis reaction it would allow for fewer steps to prepare the DNA for further processing.

The data shown the Examples demonstrate that the novel nucleotide complexes outperform nucleotide salts with monovalent cations in terms of yield and efficiency, particularly at higher concentrations of nucleotide entity. The inventors have found that the DNA synthesis is faster with the improved nucleotide complexes, meaning the time taken to produce commercial quantities of DNA is reduced. This is significant and greatly improves the furtherance of synthetic biology in therapeutic and non-healthcare applications. Notably, DNA is used as the template to manufacture large quantities of RNA, particularly mRNA, and producing DNA on a commercial scale is therefore critical to the mass-production of RNA vaccines, for example. Thus, the requirement for clean, efficient DNA manufacture on an industrial scale is currently growing exponentially. The DNA produced using the present invention may be used as a template for manufacturing SARS-Cov-2 mRNA vaccines and the like.

### SUMMARY

The present invention relates to a process for cell-free production or synthesis of DNA. The process may allow for enhanced production of DNA compared to current methodologies, i.e. an increased or greater yield, a more efficient process or the ability to perform enzymatic DNA synthesis in an environment with fewer additional components than thought possible under current methodologies. This significantly increases productivity whilst reducing the cost of synthesising DNA, particularly on a large scale.

In order to achieve a high yield at an industrial scale it is necessary to utilise high concentrations of the “building blocks” of DNA, the nucleotides (notably dNTPs). Changing the parameters of the reaction conditions alone will not increase the yields from an enzymatic reaction.

Given the provision of nucleotides to the enzymatic reaction is as a salt, increasing the quantity of nucleotides results in a significant increase in the ionic strength of the reaction mixture. The ionic strength is a function of the concentration of all ions present. Such is an important consideration as the enzymes catalysing the DNA synthesis reaction are proteins, and an increased ionic strength can result in protein unfolding and thus inactivation of enzyme activity.

Further, it can be considered that the presence of a salt can also influence the pH of the reaction mixture. Depending on the acid-base properties of the component ions, a salt can dissolve in water to provide a neutral solution (strong acid/strong base), a basic solution (weak acid/strong base) or acidic solution (strong acid/weak base). Thus, by increasing the concentration of a nucleotide salt, or any other salt to the reaction mixture (magnesium chloride as an example), this may also influence the pH and further limit the pH stabilising performance of any buffering agent present. Thus, the addition of a higher concentration of nucleotide salt, for example, may lead to sub-optimal pH control, which impacts on the enzymatic DNA synthesis notably in terms of lowering DNA yields or adversely affecting the fidelity of DNA synthesis. Therefore, there are numerous considerations to be made in relation to the “scaling-up” of enzymatic DNA manufacture, and the present inventors have devised ways to increase the yield without adversely affecting the DNA synthesis reaction.

In general, the present invention relates to enzymatic DNA synthesis using a nucleotidyltransferase, such as a polymerase enzyme or other DNA synthesising enzyme, any of which can optionally be engineered to give it particular properties.

The present invention may relate to DNA synthesis from a nucleic acid template or DNA synthesis de novo, without a template, depending on the nucleotidyltransferase used.

The present invention may relate to isothermal methods of synthesising DNA that do not require temperature to be cycled via heating and cooling during amplification, but may allow for the use of heat to initially denature the template, if present. The invention preferably relates to the use of polymerase enzymes that are capable of replicating a nucleic acid template via strand-displacement replication, independently or with the help of other enzymes.

The processes of the invention involve the use of nucleotides in the forms of complexes with associated ions, which may also be referred to herein as counter-ions. The nucleotide complex is generally present in solution, and therefore the associated counter-ion may or may not be dispersed in solution. Due to the nature of their preparation, the counter-ions may effectively be “shared” between the nucleotides, such that there is a ratio of counter-ion to nucleotide which is not a whole number. In solution with the nucleotide ionic species are divalent counter-ions and monovalent counter-ions, such that the partial or complete charge balance for each nucleotide is contributed by a mixture of monovalent and divalent cations. The use of such complexes of a nucleotide ionic species and a mixture of cation “counter-ion” species has not hitherto been seen, and is therefore a unique proposition. The provision can be such that the complex is electrically neutral, or may have a net negative charge. The nucleotide complexes may be supplied in solution, or may be dispersed in solution by adding solid nucleotide complexes to the nucleotidyltransferase in solution.

The nucleotide complexes of the present invention are in solution, and comprise a nucleotide (also described here as a nucleotide ion or ionic species) associated with at least two different positive counter-ions (cations). It is preferred that one of these counter-ions is a monovalent cation, i.e. it has a single positive charge due to the loss of one electron. It is preferred that one of these counter-ions is a divalent cation, i.e. it has a double positive charge due to the loss of two electrons. In order to increase the yield and/or efficiency of the DNA synthesis, the nucleotides are provided as a complex with a mixed counter-ion provision of monovalent and divalent cations.

It will be understood that as a nucleotide has four negative charges, it is usual to provide four positive charges, generally through 4 monovalent cations to maintain electrical neutrality, and most commercial nucleotide salts are obtained on this basis. Commercially available salts are generally limited to lithium or sodium salts. However, in the processes of the present invention, when the nucleotide complex is in solution, it is possible that less than 4 positive charges are supplied by the monovalent or divalent cations used as the counter-ions. Without wishing to be bound by theory, the inventors postulate that the remaining charges may be provided by entities such as the hydronium ion, in order to reach electrical neutrality, if required.

Accordingly there is provided a cell-free process for the enzymatic synthesis of DNA comprising the use of nucleotide complexes in solution, wherein said complex in solution comprises a nucleotide and between 0.2 and 2 divalent cations and between 0.2 and 2.5 monovalent cations per nucleotide.

It will be appreciated that the term nucleotide as used here may also be read as a nucleotide ion or nucleotide ionic species; it is the nucleotide entity without any counter-ion present.

Alternatively worded, there is provided:

A cell-free process for the enzymatic synthesis of DNA comprising obtaining a nucleotide complex in solution, wherein said complex is a nucleotide associated with between 0.2 and 2 divalent cations and between 0.2 and 2.5 monovalent cations per nucleotide, and adding a nucleotidyltransferase.

A cell-free process for the enzymatic synthesis of DNA comprising obtaining a nucleotide complex in solution, wherein said complex is a nucleotide associated with between 0.2 and 2 divalent cations and between 0.2 and 2.5 monovalent cations per nucleotide, in combination with a nucleotidyltransferase.

A cell-free process for the enzymatic synthesis of DNA using a nucleotidyltransferase comprising combining said enzyme and a nucleotide complex in solution, wherein said complex is a nucleotide associated with between 0.2 and 2 divalent cations and between 0.2 and 2.5 monovalent cations per nucleotide Also provided is a novel nucleotide complex in solution comprising a nucleotide and between 0.2 and 2 divalent cations and between 0.2 and 2.5 monovalent cations per nucleotide.

It will be appreciated that the term nucleotide as used here may also be read as a nucleotide ion or nucleotide ionic species; it is the nucleotide entity without any counter-ion present.

It will be appreciated that in solution the ions forming the complex may or may not dissociate.

It is preferred that the enzymatic DNA synthesis is for the manufacturing of DNA on a larger scale, i.e. for therapeutic or prophylactic use (stated as grams per litre reaction mixture), rather than lab-scale amplification (scale of ng to mg per litre). It is in this scaling-up of laboratory scale amplification that the present inventors have found that it is not as simple as providing more substrate and other components and finding that the yield follows suit. Thus, the process comprises the use of nucleotide complexes in general at a concentration equal or greater than 30 mM, said concentration being determined when the nucleotide complexes are combined with the nucleotidyltransferase. The admixing of the nucleotide complexes and the enzyme results in the formation of a reaction mixture. The concentration is determined in the reaction mixture in which the process is performed. Thus, the concentration of the nucleotide complexes is determined in the reaction mixture at the time that the nucleotide complexes are added. The concentration is therefore the initial concentration or concentration at the initiation of the process.

Accordingly there is provided a cell-free process for the enzymatic DNA synthesis comprising the use of nucleotide complexes at a concentration of at least 30 mM in solution, wherein said complexes each comprise a nucleotide associated with between 0.2 and 2 divalent cations and between 0.2 and 2.5 monovalent cations.

Accordingly there is provided a cell-free process for the enzymatic synthesis of DNA comprising obtaining a nucleotide complex in solution at a concentration of at least 30 mM, said complex comprising a nucleotide associated with between 0.2 and 2 divalent cations and between 0.2 and 2.5 monovalent cations per nucleotide, and adding a nucleotidyltransferase. The enzymatic DNA synthesis may involve any enzyme capable of synthesising DNA, notably a nucleotidyltransferase, the definition of which herein includes all enzymes capable of transferring a nucleotide to the end of a nascent polynucleotide chain, either on the basis of a template or de novo. The nucleotidyltransferase may include a polymerase or a modified polymerase, such as a DNA polymerase or an RNA polymerase. The polymerase may be from any of the known families of DNA polymerase, including families A, B, C, D, X, Y and RT. An example of a DNA polymerase from family X is terminal deoxynucleotidyl transferase.

The nucleotidyltransferase may be present in solution, and the nucleotide complexes can be added as a solid preparation to the enzyme in solution, for example as a lyophilised powder.

The enzymatic DNA synthesis may occur de novo without the use of a template.

The enzymatic DNA synthesis may involve a template, such as a nucleic acid template, including a DNA template.

The enzymatic DNA synthesis may take place in a reaction mixture, comprising the components described here.

Alternatively written, there is provided a cell-free process for synthesising DNA in solution comprising contacting a template with at least one nucleotidyltransferase in the presence of one or more nucleotide complexes, wherein said nucleotide complexes comprise a nucleotide associated with between 0.2 and 2 divalent cations and between 0.2 and 2.5 monovalent cations per nucleotide. Optionally the concentration of said nucleotide complexes is at least 30 mM, preferably 40 mM.

Alternatively put, the nucleotide complexes include a mixture of divalent and monovalent cations, along with the nucleotide itself. Accordingly there is provided a cell-free process for synthesising DNA comprising contacting a template with at least one nucleotidyltransferase in the presence of one or more nucleotide complexes to form a reaction mixture, wherein said nucleotide complexes are present at a concentration of at least 30 mM and comprise a nucleotide associated with between 0.2 and 2 divalent cations and between 0.2 and 2.5 monovalent cations.

It is preferred that when the concentration of the nucleotide or nucleotide complex is referred to, this is the concentration of nucleotide (or complex thereof) when the process begins, i.e. the starting or initial concentration of nucleotide (or nucleotide complex). Thus, it is the concentration after addition to the reaction mixture. It will be appreciated that addition of other components can be made during the process; such additions may dilute the concentration of the nucleotides/nucleotide complexes, unless further nucleotides/nucleotide complexes are supplied to replenish the concentration. Further, since the nucleotide/nucleotide complexes will be used or consumed by the process, i.e. the DNA synthesis reaction, the concentration of the nucleotide/nucleotide complexes will fall as the process progresses. In certain embodiments, further nucleotides/nucleotide complexes may be added as the process progresses in order to replenish substrates for the enzymatic reaction.

The inventors have surprisingly found that if the nucleotide complexes include a mixture of monovalent and divalent cations that the yield can be further improved. This further improvement is compared to a conventional nucleotide salt with 4 monovalent cations. Comparative Examples are included here. This effect is most notable for higher concentrations of nucleotide complex, for example above 30 mM. The inventors have also noted that the association of magnesium or manganese cations with the nucleotide complex means that no additional magnesium or manganese is required in the reaction mixture in order to synthesize DNA. This is beneficial in reducing components and therefore cost.

Convention dictates, for example, that magnesium (a divalent cation) is present in DNA synthesis reactions at a minimum ratio of at least 1:1 with the nucleotides. This is because magnesium may be required at the active site of some nucleotidyltransferase enzymes; it may form a complex with the nucleotide prior to incorporation and further may form its own salt with the phosphate ionic species released during DNA synthesis. The benefit of including magnesium or manganese in the nucleotide complex is that this reduces or removes the requirement for additional magnesium or manganese. It assists to maintain an approximate 1:1 ratio (or below 1:1 ratio) of magnesium or manganese to nucleotide, which the inventors have previously identified as desirable in large scale DNA synthesis reactions. This is important, since reducing the components included in the DNA synthesis notably reduces costs, but furthermore a higher concentration (greater than 1:1) of magnesium is related to a decreased fidelity in DNA synthesis.

The divalent cations associated with the nucleotide in the complex may comprise one or more metals selected from the list consisting of: magnesium (Mg²⁺), beryllium (Be²⁺), calcium (Ca²), strontium (Sr²⁺), manganese (Mn²⁺) or zinc (Zn²⁺), preferably Mg²⁺ or Mn²⁺. The ratio between the divalent metal cations and the nucleotide (nucleotide ion or nucleotide ionic species) may be about 1:1 in solution, but is preferably between 0.2:1 and 2:1, optionally 0.5:1 to 1.5:1. Ratios lower than 1:1 are desirable and are preferable in DNA synthesis since ratios higher than 1:1 may lead to some infidelity in DNA synthesis. The provision of the divalent cations in relation to the nucleotide complex may therefore reduce or remove the need to add additional divalent cations to the reaction mixture. However, should further be required, these divalent cations may be provided to the enzymatic DNA synthesis in the form of any suitable salt.

Further, the method developed here by the present inventors can be performed in a large range of conditions with respect to the other components present. These conditions range from a conventional level of buffering to the provision of no further buffering agents, effectively performing the reaction with the required components in water. Increasing the concentration of a buffering agent may increase buffering capacity to directly improve pH control but chemical buffers can also chelate a range of metal ions including magnesium ions and may adversely interfere with the balance of mono- and divalent cations required for optimal DNA yields. Using concentrations of buffering agents at the lowest possible levels while balancing other essential reaction components to maintain pH within an acceptable band for optimal DNA production may, therefore, be desirable. Those skilled in the art will appreciate that some of the counter-ions proposed here may have their own buffering ability, or entities released or produced during DNA synthesis (for example pyrophosphates and phosphates) may also help buffer against excessive pH change.

The required components, regardless of buffering agent provision, may include the DNA synthesising enzyme (nucleotidyltransferase), such as a polymerase, the nucleotide complexes, with optional additional components required depending on the conditions of the reaction, selected from divalent metal cations provided as a salt, a template, a denaturing agent, a pyrophosphatase, or one or more primers/primase. These components may form the reaction mixture. Thus, in its most basic form, the reaction mixture is simply the nucleotide complexes plus a nucleotidyltransferase. It will be understood that it is desirable that the reaction mixture does not contain superfluous ionic species, since such entities may undesirably affect the DNA synthesis. Other than the ions present in the nucleotide complex, other ions may be present in minimal amounts, such as in denaturing agents (for example sodium or potassium hydroxide) or in buffering agents. It may be necessary to further supplement magnesium or manganese salts, depending on the nucleotide complex selected and enzyme involved in the reaction. It is preferred that the concentration of “additional ions” in the reaction mixture, such as at the start of the reaction, may be kept to a minimal level, for example less than 50 mM, less than 40 mM, less than 30 mM, less than 20 mM, or less than 10 mM. Such additional ions are ions other than those supplied with the nucleotide complex or generated therefrom during the course of the reaction.

Thus, the provision to the process, i.e. the reaction mixture, of nucleotides as complexes with a mixed counter ion provision is advantageous, since this surprisingly allows for improved DNA yield and/or an improved efficiency of conversion of the nucleotides into DNA. These improvements may be compared to an analogous reaction mixture where all the nucleotides are supplied as conventional salts with the requisite monovalent cation alone. The provision of novel nucleotide complexes instead of nucleotide salts conventionally used has some further surprising advantages, such as the ability to lower the concentration of buffering agents in the reaction mixture, in some instances to zero, and/or to lower, decrease or remove entirely the requirement for the additional provision of a divalent cation co-factor for the reaction mixture, most notably magnesium, which is usually added as a salt. Given that this salt may no longer be required, the present invention has the effect of reducing the ionic strength of the reaction mixture, since if the divalent cation salt is not added, there is no provision to the reaction mixture of the associated anion (e.g. MgCl₂— therefore the addition of two chloride ions is avoided). The ionic strength of a solution is a measure of the concentration of ions in that solution. Ionic compounds, when dissolved in water, dissociate into ions. As used herein, the unit of measurement is molar (mol/L). The inventors postulate that a reduction in the ionic strength of the reaction mixture may be beneficial to the process. It is possible that the monovalent cations provided with the nucleotide entity are inhibitory to the process in high concentrations. Alternatively or additionally, in a standard process, magnesium or manganese is supplied as a salt, and high concentrations of the associated anion from this salt may also be inhibitory to the process, for example the chloride ion. Furthermore, should it be desirable to manipulate the DNA produced using the invention, the inventors have seen that enzymes introduced into the reaction mixture, such as enzymes that cleave and ligate target sequences (DNA processing enzymes), prefer the conditions of lower ionic strength, since these are generally added at the end of the DNA synthesis reaction, when the ionic strength may have increased significantly with conventional nucleotide and divalent salts. Thus, the product from such a DNA synthesis reaction may be suitable for further processing enzymatically (such as use as a template to produce RNA using RNA polymerase).

In one aspect, a template directs the enzymatic DNA synthesis in the processes. This template may be any nucleic acid template, for example a DNA or RNA template. The template may be a natural nucleic acid, an artificial nucleic acid or a combination of the two. The amplification of the template is preferably via strand-displacement. The amplification of the template is preferably isothermal, i.e. there is no requirement to cycle between low and high temperatures to progress the amplification. In this scenario, heat may be used at the start to denature the template, if required, or the template may be denatured by chemical means. However, once the template has been denatured, if appropriate, to allow any primers or indeed primase enzyme to enter between double stranded templates, the temperature may be maintained at a range of temperatures that do not affect the denaturation of the template and product. Isothermal temperature conditions require that the reaction is not heated to a point where the template and products denature (compared to PCR which requires cycles of heat to denature the template and product). Generally such reactions are performed at a constant temperature, depending on the preference of the enzyme itself. The temperature may be any suitable temperature for the enzyme.

The cell-free process preferably involves amplification of the template via strand displacement replication. This synthesis releases a single stranded DNA, which may in turn be copied into double stranded-DNA, using a polymerase. The term strand displacement describes the ability to displace downstream DNA encountered during synthesis, wherein the polymerase opens the double-stranded DNA in order to extend the nascent single strand. DNA polymerases with varying degrees of strand displacement activity are available commercially. Alternatively, strand displacement can be achieved by supplying a DNA polymerase and a separate helicase. Replicative helicases may open the duplex DNA and facilitate the advancement of the leading-strand polymerase.

Independently, optional features of any aspect of the invention may be: The template may be circular. The DNA may be synthesized by amplification of a template, optionally by strand displacement replication. The strand displacement amplification of said DNA template may be carried out by rolling circle amplification (RCA). The polymerase may be Phi29, or a variant thereof. The amplification of DNA may be isothermal amplification, i.e. at a constant temperature. A primer or primase may be used to initiate amplification. A nickase may be used to generate a primer “in situ” on a double stranded circular template. The one or more primers may be random primers. A pair or set of primers may be used. The synthesised DNA may comprise concatamers comprising tandem units of DNA sequence amplified from the DNA template. The DNA template may be a closed linear DNA; preferably the DNA template is incubated under denaturing conditions to form a closed circular single stranded DNA.

The quantity of DNA that may be synthesised is equal to or higher than 3 g/litre of reaction mixture, notably 16 g/l or more, preferably up to 25 g/l and beyond.

The amount of DNA that may be synthesised may exceed 60% of the calculated maximum yield for the reaction mixture. Preferably, the amount of DNA that may be synthesised may exceed 80% of the calculated maximum yield. The calculated maximum yield is based upon the theoretical yield should all nucleotides be incorporated into a product, and this can be calculated by those skilled in the art.

The efficiency of DNA synthesis from the nucleotides (or nucleotide complexes) may be described as the percentage of nucleotides or complexes thereof supplied to the reaction mixture which are successfully incorporated into a product over the course of the reaction.

The speed of the DNA synthesis may also be improved by the present invention, reducing the time taken to make a significant DNA yield.

The cell-free process requires at least one nucleotide complex. One or more further nucleotide complexes may then be added. Any suitable number of phosphate groups may be present, as required. However, it is preferred that the nucleotides/nucleotide complexes or further nucleotides/nucleotide complexes are deoxyribonucleoside triphosphates (dNTPs) or a derivative or modified version thereof. The nucleotides or further nucleotides are one or more of deoxyadenosine triphosphate (dATP), deoxyguanosine triphosphate (dGTP), deoxycytidine triphosphate (dCTP), deoxythymidine triphosphate (dTTP) and derivatives thereof. The nucleotides or further nucleotides are provided as complexes thereof. Each individual nucleotide complex may, but is not required to, be charged balanced with various cations providing 4 positive charges to maintain electrical neutrality. The nucleotide complexes used in the process may include one or more monovalent cations, i.e. one or more species of monovalent cation and one or more divalent cations, i.e. one or more species of divalent cation. It will be appreciated that these may dissociate in solution, and because of this, the number of cations associated with each nucleotide complex does not need to be a whole number, since in solution the ions may dissociate. The nucleotide complex may be considered to be a salt if the charges are entirely balanced.

In general, the inventors have mixed two different nucleotide complexes (one with solely divalent cation(s) and the other with solely monovalent cations) together in order to prepare the nucleotide complexes used in the method of the invention. The nucleotide complex may each independently comprise a complex where not all of the negative charges are balanced. This has several advantages. Nucleotides complexed with divalent cations have a low solubility, and therefore are not in routine use for any application. Particular issues are seen with nucleotides complexed with magnesium ions; they are not in solution and are unusable in their current form. However, when mixed with nucleotide complexes associated with a monovalent cation or cations, the mixture is soluble and forms a solution. Therefore, this provides an elegant way of utilising a previously desired but unworkable nucleotide complex.

The monovalent ion may be a sole species of ion, or a mixture of different species of ion. The divalent ion may be a sole species of ion or a mixture of different species of ion.

0.2 to 2.5 monovalent cations are generally present in or associated with the nucleotide complex according to the present invention. This range includes 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4 and 2.5 monovalent ions per nucleotide complex. It is possible that the ions are shared between the nucleotide ions. It is, however, possible that the level of monovalent cation could be reduced to minimal levels or even complete absence, by the alternative use of buffering agents to ensure solubility of the nucleotide complexes associated with divalent cations. This would have further beneficial effects, since the level of monovalent cation associated with the nucleotide complex could be taken lower than 0.2:1 and may even reach 0.1:1 or zero. Suitable buffering agents for this embodiment include those that are not capable or complexing the metal cations present in the reaction mixture, since protons are released when a buffer forms complexes with metal ions. For example, buffers HEPES and HEPPS both have negligible metal ion affinity, or BES, which does not interact with magnesium.

It is preferred that the concentration of nucleotides or complexes thereof in the process, i.e. in the reaction mixture, may be more than 30 mM and up to at least 300 mM. Such concentrations are important in the production of higher yields of DNA, which in the case of the two concentrations given can be as high as 9.75 g/l to 97.5 g/l. It is preferred that the concentration of nucleotide or complexes thereof stated is at the start of the process, i.e. is the starting or initial concentration of nucleotides or complexes thereof in the reaction mixture, which also includes the enzyme necessary for DNA synthesis. Subsequent addition of further components may reduce this concentration, and their use by the DNA synthesis enzyme will also reduce the concentration from the starting concentration. Those skilled in the art will be aware of how to calculate the concentration of nucleotides/nucleotide complexes as the process is prepared, based upon the volume of the other components and the stock nucleotide complex solution/powder used.

It should be noted that in the art of DNA synthesis or amplification, the term “nucleotide” is used when the author means “nucleotide salt”, since the provision and utilisation in DNA synthesis of nucleotides without any form of counter-ion is not currently possible. The process may be a batch process or a continuous flow process. The batch may be a closed batch (i.e. all of the reaction components are provided at the start of the DNA synthesis) or further components can be supplied to the reaction as required during the process, such as described in WO2016/034849, incorporated herein by reference. Should further additions be required, this will dilute the concentration of the nucleotide or nucleotide complexes, unless further nucleotide complexes are added to replenish or increase the concentration.

The present inventors have found that each of the different counter-ions may add a particular characteristic to the enzymatic DNA synthesis reaction. For example the use of ammonium ions in nucleotide complexes may result in the use of some higher concentrations of nucleotides.

The enzymatic cell-free synthesis of DNA with such ions can be carried out in minimal buffering agents, in which no additional salts which have been shown to enhance DNA synthesis or assist in primer binding, or detergents, are added. This minimal buffer may comprise an agent (a buffering agent) to stabilise the pH. The minimal buffering agent may contain a small amount of cations provided by the presence of a chemical used to denature the template, such as sodium, potassium or ammonium hydroxide. In the Examples, 5 mM sodium or potassium hydroxide was used as a denaturant, but this concentration may be altered to suit the conditions of the reaction, within the skill of those used to performing DNA denaturation, and amounts of sodium, potassium or ammonium hydroxide could be provided from 2.5 mM, up to 5 mM, up to 10 mM, 15 mM, 20 mM or 25 mM or more may be employed dependent upon the nature of the template. Therefore, the reaction mixture may contain a small or minimal amount of cations and anions which were not originally associated with the nucleotide complex.

Further advantages are described below.

### BRIEF DESCRIPTION OF THE DRAWINGS

The present invention will be described further below with reference to exemplary embodiments and the accompanying drawings, in which:

FIGS. 1A to 1B are plots showing results obtained with experiments using varied starting concentrations of nucleotide salts for either sodium alone (dNTP:4Na⁺)(continuous line) or a mixed sodium/magnesium dNTP (dNTP:2Na⁺/Mg²⁺)(dotted line). The plot depicts the reaction yields in g/l (A) and the reaction efficiency as a % (B) for each type of dNTP plotted vs the start dNTP concentration used. FIG. 1C shows DNA produced after 1 week of RCA, with more viscous DNA solutions corresponding to higher yields remaining attached to the base of the Eppendorf tube when inverted.

FIGS. 2A to 2B are plots showing results obtained with experiments using varied starting concentrations of nucleotide salts for either ammonium alone (dNTP:4NH₄+) (continuous line) or a mixed ammonium/magnesium dNTPs (dNTP: 2NH₄⁺/Mg²⁺)(dotted line). The plot depicts the reaction yields in g/l (A) and the reaction efficiency as a % (B) for each type of dNTP plotted vs the start dNTP concentration used. FIGS. 2C and 2D shows the viscosity of the DNA produced after 18 hrs and 106 hrs of RCA respectively.

FIGS. 3A to 3C are plots showing the results obtained using varied starting concentrations of nucleotide salts for either a monovalent dNTP (dotted line) or a mixed monovalent/magnesium dNTP (continuous line). The plot depicts the concentration (in mM) of the dNTP entity at the start vs reaction yields in g/l. The data shown is the peak yield for those dNTPs over the course of the reaction. FIG. 3A shows the peak yield results for ammonium dNTPs (dotted line) and mixed ammonium/magnesium dNTPs (continuous line) over the course of 6 days. FIG. 3B shows the peak yield results for potassium dNTPs (dotted line) and mixed potassium/magnesium dNTPs (continuous line) over the course of 6 days. FIG. 3C shows the peak yield results for caesium dNTPs (dotted line) and mixed caesium/magnesium dNTPs (continuous line) over the course of 5 days.

FIG. 4 is a chart showing the results for obtained using varied starting concentrations of nucleotide salts for either a monovalent dNTP or a mixed monovalent/magnesium dNTP. The chart depicts the start concentration of the dNTP entity indicated at the start vs peak reaction yields in g/l. Each dNTP entity is tested in starting concentrations of 5, 10, 20, 30, 80, 100 and 120 mM.

FIGS. 5A and 5B are plots showing the results obtained using varied starting concentrations of nucleotide salts for either a monovalent dNTP (dotted line and dashed line) or a mixed monovalent/magnesium dNTP (continuous line). The plot depicts the concentration (in mM) of the dNTP entity at the start vs reaction yields in g/l. The dotted line indicates that the monovalent dNTP was supplemented with magnesium acetate and the dashed line indicates that the monovalent dNTP was supplemented with magnesium chloride. The mixed dNTP was not supplemented with further magnesium salts. FIG. 5A shows the peak yield results for ammonium dNTPs (dotted line and dashed line) and mixed ammonium/magnesium dNTPs (continuous line). FIG. 5B shows the peak yield results for sodium dNTPs (dotted line and dashed line) and mixed sodium/magnesium dNTPs (continuous line).

FIGS. 6A and 6B are charts showing the results obtained using varied starting concentrations of nucleotide salts for either a monovalent dNTP (supplemented with either magnesium chloride or magnesium acetate) or a mixed monovalent/magnesium dNTP (no additional magnesium). The chart depicts the concentration (in mM) of the dNTP entity at the start vs reaction yields in g/l. FIG. 6A shows the peak yield results for ammonium dNTPs and mixed ammonium/magnesium dNTPs. FIG. 6B shows the peak yield results for sodium dNTPs and mixed sodium/magnesium dNTPs.

FIGS. 7A and 7B are charts showing the results obtained using varied starting concentrations of ammonium: magnesium dNTPs (2 ammonium: 1 magnesium) in either 30 mM Tris buffer pH 8.0 or in water. The bar charts are a plot of starting concentration of dNTP (in mM with each day of measurement indicated) vs yield of DNA obtained in g/l. FIG. 7A depicts the results in 30 mM Tris buffer pH 8.0 whilst FIG. 7B depicts the results in water (no added buffering agent).

### DETAILED DESCRIPTION

The present invention relates to cell-free processes for large scale synthesis of DNA. The processes of the invention may allow for a high throughput synthesis of DNA.

The deoxyribonucleic acid (DNA) synthesised according to the present invention can be any DNA molecule. The DNA may be single stranded or double stranded. The DNA may be linear. The DNA may be processed to form circles, particularly minicircles, single stranded closed circles, double stranded closed circles, double stranded open circles, or closed linear double stranded DNA. The DNA may be allowed to form, or processed to form a particular secondary structure, such as, but not limited to hairpin loops (stem loops), imperfect hairpin loops, pseudoknots, or any one of the various types of double helix (A-DNA, B-DNA, or Z-DNA). The DNA may also form hairpins and aptamer structures.

The DNA synthesised may be of any suitable length. Lengths of up to or exceeding 77 kilobases may be possible using the processes of the invention. More particularly, the length of DNA which may be synthesised according to the processes of the invention may be in the order of up to 60 kilobases, or up to 50 kilobases, or up to 40 kilobases, or up to 30 kilobases. Preferably the DNA synthesised may be 100 bases to over 77 kilobases, 500 bases to 60 kilobases, 200 bases to 20 kilobases, more preferably 200 bases to 15 kilobases, most preferably 2 kilobases to 15 kilobases.

The amount of DNA synthesised according to the processes of the present invention may exceed 9.75 g/l. It is preferred that the amount of DNA synthesised is greater than 3, 4, 5, 6, 7, 8, 9, 10, 11, 12, 13, 14, 15, 16, 17, 18, 19, 20, 21, 22, 23, 24, 25, 26, 27, 28, 29 or 30 g/l or more. A preferred amount of DNA synthesised is 5 g/l. The amount of DNA produced may be described as industrial or commercial quantities, on a large-scale or mass production. The DNA produced by the processes of the invention may be uniform in quality, namely in DNA length and sequence. The processes may thus be suitable for large scale synthesis of DNA. The process may be uniform in terms of fidelity of synthesis.

Alternatively, the amount of DNA produced in the synthesis reaction may be compared to the theoretical maximum yield which would be achieved if 100% nucleotides were incorporated into the synthesised DNA. The methods of the invention not only improve the total yield obtained, but also the efficiency of the process, meaning that more of the supplied nucleotides are incorporated into the synthesised DNA product than in previous methods. Yields obtainable by the methods of the invention exceed 50% of the theoretical maximum, up to and exceeding 90% of the theoretical maximum. Therefore, the proportion of the theoretical maximum yield achieved by methods of the invention include 50%, 55%, 60%, 65%, 70%, 75%, 80%, 85%, 90% and 95% or greater. Conventionally, using commercially available nucleotide salts, yields achieved could be disappointing, due to effects of the ions that may be inhibitory to the process.

The DNA is synthesised in an enzymatic reaction. This enzymatic synthesis may involve the use of any DNA synthesising enzyme, a nucleotidyltransferase, capable of adding a nucleotide to a nascent polynucleotide chain, most notably a polymerase enzyme or a modified polymerase enzyme. These are discussed further below. The DNA synthesis may be de novo and not require a template. The enzymatic synthesis may also require the use of a template for the DNA synthesis. This template can be any suitable nucleic acid depending on the polymerase, but is preferably a DNA template.

The template may be any suitable template, merely providing the instructions for the synthesis of the DNA by including a particular sequence. The template may be single stranded (ss) or double stranded (ds). The template may be linear or circular. The template may include natural, artificial or modified bases or a mixture thereof.

The template may comprise any sequence, either naturally derived or artificial.

The template may be of any suitable length. Particularly, the template may be up to 60 kilobases, or up to 50 kilobases, or up to 40 kilobases, or up to 30 kilobases. Preferably the DNA template may be 10 bases to 100 bases, 100 bases to 60 kilobases, 200 bases to 20 kilobases, more preferably 200 bases to 15 kilobases, most preferably 2 kilobases to 15 kilobases.

The template may be provided in an amount sufficient for use in the process by any method known in the art. For example, the template may be produced by PCR.

The whole or a selected portion of the template may be amplified in the process.

The template may comprise a sequence for expression. The DNA may be for expression in a cell (i.e. a transfected cell in vitro or in vivo), or may be for expression in a cell free system (i.e. protein synthesis). The sequence for expression may be for therapeutic purposes, i.e. gene therapy or a DNA vaccine. The sequence for expression may be a gene, and said gene may encode a DNA vaccine, a therapeutic protein and the like. The sequence may comprise a sequence which is transcribed into an active RNA form, i.e. a small interfering RNA molecule (siRNA). The sequence may comprise a sequence which is transcribed into mRNA, most particularly an mRNA for producing a vaccine.

If required, the template may be contacted with at least one polymerase, as described below.

The enzymatic DNA synthesis reaction may require at least one DNA synthesis enzyme (a nucleotidyltransferase). Preferably, the DNA synthesis enzyme is a polymerase. Polymerases link together nucleotides to form a DNA polymer. One, two, three, four or five different enzymes and/or polymerases may be used. The polymerase may be any suitable polymerase from any family of polymerases, such that it synthesises polymers of DNA. The polymerase may be a DNA polymerase. Any DNA polymerase may be used, including any commercially available DNA polymerase. Two, three, four, five or more different DNA polymerases may be used, for example one which provides a proofreading function and one or more others which do not. DNA polymerases having different mechanisms may be used e.g. strand displacement type polymerases and DNA polymerases replicating DNA by other methods. A suitable example of a DNA polymerase that does not have strand displacement activity is T4 DNA polymerase. Template-independent polymerases may be used, such as terminal transferases.

Modified polymerases may also be used. These may have been engineered to modify their characteristics, such as to remove their dependency upon a template, to change their temperature dependency or to stabilise the enzyme for use in vitro.

A polymerase may be highly stable, such that its activity is not substantially reduced by prolonged incubation under process conditions. Therefore, the enzyme preferably has a long half-life under a range of process conditions including but not limited to temperature and pH. It is also preferred that a polymerase has one or more characteristics suitable for a manufacturing process. The polymerase preferably has high fidelity, for example through having proofreading activity. Furthermore, it is preferred that a polymerase displays one or more of: high processivity, high strand-displacement activity and a low Kₘ for dNTPs and DNA. A polymerase may be capable of using circular and/or linear DNA as template. The polymerase may be capable of using dsDNA or ssDNA as a template. It is preferred that a polymerase does not display DNA exonuclease activity that is not related to its proofreading activity. Further, the polymerase may be capable of using an alternative nucleic acid as a template.

The skilled person can determine whether or not a given polymerase displays characteristics as defined above by comparison with the properties displayed by commercially available polymerases, e.g. Phi29 (New England Biolabs, Inc., Ipswich, Mass., US), Deep Vent® (New England Biolabs, Inc.), Bacillus stearothermophilus (Bst) DNA polymerase I (New England Biolabs, Inc.), Klenow fragment of DNA polymerase I (New England Biolabs, Inc.), M-MuLV reverse transcriptase (New England Biolabs, Inc.), VentR®(exo-minus) DNA polymerase (New England Biolabs, Inc.), VentR® DNA polymerase (New England Biolabs, Inc.), Deep Vent® (exo-) DNA polymerase (New England Biolabs, Inc.) and Bst DNA polymerase large fragment (New England Biolabs, Inc.). Where a high processivity is referred to, this typically denotes the average number of nucleotides added by a polymerase enzyme per association/dissociation with the template, i.e. the length of nascent extension obtained from a single association event.

Strand displacement-type polymerases are preferred. Preferred strand displacement-type polymerases are Phi29, Deep Vent and Bst DNA polymerase I or variants of any thereof. “Strand displacement” describes the ability of a polymerase to displace complementary strands on encountering a region of double stranded DNA during synthesis. The template is thus amplified by displacing complementary strands and synthesising a new complementary strand. Thus, during strand displacement replication, a newly replicated strand will be displaced to make way for the polymerase to replicate a further complementary strand. The amplification reaction initiates when a primer or the 3′ free end of a single stranded template anneals to a complementary sequence on a template (both are priming events). When DNA synthesis proceeds and if it encounters a further primer or other strand annealed to the template, the polymerase displaces this and continues its strand elongation. The strand displacement may release single stranded DNA which can act as a template for more priming events. The priming of the newly released DNA may lead to hyper-branching, and a high yield of products. It should be understood that strand displacement amplification methods differ from PCR-based methods in that cycles of denaturation are not essential for efficient DNA amplification, as double-stranded DNA is not an obstacle to continued synthesis of new DNA strands. Strand displacement amplification may only require one initial round of heating, to denature the initial template if it is double stranded, to allow the primer to anneal to the primer binding site, if a primer is used. Following this, the amplification may be described as isothermal, since no further heating or cooling is required. In contrast, PCR methods require cycles of denaturation (i.e. elevating temperature to 94 degrees centigrade or above) during the amplification process to melt double-stranded DNA and provide new single-stranded templates. During strand displacement, the polymerase will displace strands of already synthesised DNA. Further, it will use newly synthesised DNA as a template, ensuring rapid amplification of DNA.

A strand displacement polymerase used in a process of the invention preferably has a processivity of at least 20 kb, more preferably, at least 30 kb, at least 50 kb, or at least 70 kb or greater. In one embodiment, the strand displacement DNA polymerase has a processivity that is comparable to, or greater than phi29 DNA polymerase.

Strand displacement replication is, therefore, preferred. During strand displacement replication, the template is amplified by displacing already replicated strands, which have been synthesised by the action of the polymerase, in turn displacing another strand, which can be the original complementary strand of a double stranded template, or a newly synthesised complementary strand, the latter synthesised by the action of a polymerase on an earlier primer annealed to the template. Thus, the amplification of the template may occur by displacement of replicated strands through strand displacement replication of another strand. This process may be described as strand displacement amplification or strand displacement replication.

A preferred strand displacement replication process is Loop-mediated isothermal amplification, or LAMP. LAMP generally uses 4-6 primers recognizing 6-8 distinct regions of the template DNA. In brief, a strand-displacing DNA polymerase initiates synthesis and 2 of the primers form loop structures to facilitate subsequent rounds of amplification. An inner primer containing sequences of the sense and antisense strands of the target DNA initiates LAMP. The following strand displacement DNA synthesis primed by an outer primer releases a single-stranded DNA. This serves as template for DNA synthesis primed by the second inner and outer primers that hybridize to the other end of the target, which produces a stem-loop DNA structure. In subsequent LAMP cycling one inner primer hybridizes to the loop on the product and initiates displacement DNA synthesis, yielding the original stem-loop DNA and a new stem-loop DNA with a stem twice as long. Modified LAMP procedures can also be adopted, where fewer internal primers are required.

A preferred strand displacement replication process is rolling circle amplification (RCA). The term RCA describes the ability of RCA-type polymerases to continuously progress around a circular DNA template strand whilst extending a hybridised primer. The “primer” may be added, created by a primase or generated by nicking one strand of a double stranded template. This amplification leads to formation of linear single stranded products with multiple repeats of amplified DNA. The sequence of the circular template (a single unit) is multiply repeated within a linear product. For a circular template, the initial product of strand displacement amplification is a single stranded concatamer, which is either sense or antisense, depending on the polarity of the template. These linear single stranded products serve as the basis for multiple hybridisation, primer extension and strand displacement events, resulting in formation of concatameric double stranded DNA products, again comprising multiple repeats of amplified DNA. There are thus multiple copies of each amplified “single unit” DNA in the concatameric double stranded DNA products. RCA polymerases are particularly preferred for use in the processes of the present invention. The products of RCA-type strand displacement replication processes may require processing to release single unit DNAs. This is desirable if single units of DNA are required. Typical strand displacement conditions using Phi29 DNA polymerase include high levels of magnesium ions, for example 10 mM magnesium (normally as a chloride salt) in combination with 0.2 to 4 mM nucleotides (when presented as typical lithium or sodium salts).

In order to allow for amplification, according to some aspects one or more primers may also be required by the enzymatic DNA synthesis. If no template is used, the primers allow for a starting point for DNA synthesis and are designed to begin the synthesis reaction. If a template is used, the primers may be non-specific (i.e. random in sequence) or may be specific for one or more sequences comprised within the template. Alternatively, a primase enzyme may be supplied to generate the primer de novo. If the primers are of random sequence they allow for non-specific initiation at any site on the template. This allows for high efficiency of amplification through multiple initiation reactions from each template strand. Examples of random primers are hexamers, heptamers, octamers, nonamers, decamers or sequences greater in length, for example of 12, 15, 18, 20 or 30 nucleotides in length. A random primer may be of 6 to 30, 8 to 30 or 12 to 30 nucleotides in length. Random primers are typically provided as a mix of oligonucleotides which are representative of all potential combinations of e.g. hexamers, heptamers, octamers or nonamers in the template.

In one embodiment, the primers or one or more of the primers are specific. This means they have a sequence which is complementary to a sequence in the template from which initiation of amplification is desired. In this embodiment, a pair of primers may be used to specifically amplify a portion of the DNA template which is internal to the two primer binding sites. Alternatively, a single specific primer may be used. A set of primers may be employed.

Primers may be any nucleic acid composition. Primers may be unlabelled, or may comprise one or more labels, for example radionuclides or fluorescent dyes. Primers may also comprise chemically modified nucleotides. For example, the primer may be capped in order to prevent initiation of DNA synthesis until the cap is removed, i.e., by chemical or physical means. Primer lengths/sequences may typically be selected based on temperature considerations i.e. as being able to bind to the template at the temperature used in the amplification step. Primers may be RNA primers, such as those synthesized by a primase.

In certain aspects, the contacting of the template with the synthesis enzyme and one or more primers may take place under conditions promoting annealing of primers to the template. The conditions include the presence of single-stranded nucleic acid allowing for hybridisation of the primers. The conditions conventionally also include a temperature and buffer allowing for annealing of the primer to the template. Appropriate annealing/hybridisation conditions may be selected depending on the nature of the primer. An example of conventional annealing conditions, which may be used in the present invention include a buffer comprising 30 mM Tris-HCl pH 7.5, 20 mM KCl, 8 mM MgCl₂. In the Examples, the reactions with the nucleotide complexes of the invention are performed in 30 mM Tris pH 8.0 as a buffering agent alone. However, the present inventors have described conditions herein with reduced buffer and divalent metal ion components that still allow for primer binding and these are discussed further below. The annealing may be carried out following denaturation using heat followed by gradual cooling to the desired reaction temperature.

However, amplification using strand displacement replication can also take place without a primer, and thus requires no hybridisation and primer extension to occur. Instead, the single stranded template self-primes by forming hairpins, which have a free 3′ end available for extension. The remaining steps of the amplification remain the same. Alternatively, a double stranded template can be nicked to allow for strand displacement replication to use one strand of the template itself as a primer. Those skilled in the art are aware of all methods for providing initiation of amplification from a template.

The template and/or polymerase are also contacted with nucleotides, as nucleotide complexes as defined herein. The combination of template, nucleotidyltransferase and nucleotide complexes may be described as forming a reaction mixture. The reaction mixture may also comprise one or more primers or a primase. The reaction mixture may independently also include one or more divalent metal cations, should sufficient not be supplied with the nucleotide complexes. The reaction mixture may further comprise a chemical denaturant. Such denaturants can be potassium, ammonium or sodium hydroxide. The reaction mixture may further comprise additional enzymes, such as a helicase or a pyrophosphatase. The reaction mixture may contain pH buffering agents, and in some aspects, it contains no additionally added pH buffering agents.

A nucleotide is a monomer, or single unit, of nucleic acids, and nucleotides are composed of a nitrogenous base, a five-carbon sugar (ribose or deoxyribose), and at least one phosphate group. Any suitable nucleotide may be used.

The nucleotides are present as complexes, and are thus associated with a mixture of divalent and monovalent cations. Monovalent cations are ionic species with a single positive charge, and may be a metal ion or a polyatomic ion, such as an oxonium ion. Divalent cations are ionic species with a double positive charge, and may be a metal ion or a polyatomic ion.

A counter-ion is the ion that accompanies or is associated with an ionic species (the nucleotide in the present invention) in order to partially or completely balance the charge on the ionic species.

A complex is generally understood to be a molecular entity formed by loose association involving two or more component molecular entities (ionic or uncharged), or the corresponding chemical species. A complex may be either an ion or an electrically neutral molecule, formed by the union of simpler substances (as compounds or ions) and held together by forces that are chemical (i.e., dependent on specific properties of particular atomic structures) rather than physical. The bonding between the components is normally weaker than in a covalent bond.

The nucleotide complexes may include monovalent metal ions, including but not limited to alkali metals (group 1): lithium (Li⁺), sodium (Na⁺), potassium (K⁺), rubidium (Rb⁺), caesium (Cs⁺) or francium (Fr⁺). Alternatively or additionally, the monovalent metal ion may be a transition metal (Group 11): copper (Cu⁺), silver (Ag⁺), gold (Au⁺) or roentgenium (Rg⁺). The alkali metals are preferred, and thus the preferred counter-ion may be lithium (Li⁺), sodium (Na⁺), potassium (K⁺), rubidium (Rb⁺), caesium (Cs⁺) or francium (Fr⁺).

The nucleotide complexes may include polyatomic monovalent ions. A polyatomic ion is an ion that contains more than one atom. This differentiates polyatomic ions from monatomic ions, which contain only one atom. Exemplary monovalent polyatomic cations include oxonium ions. An oxonium ion is any oxygen cation with three bonds. The simplest oxonium ion is the hydronium ion H₃O. Other notable oxonium ions include ammonium (NH₄⁺) and ionic derivatives of ammonium.

Derivatives of ammonium are also encompassed, and a non-limiting, exemplary list of these includes: monoalkyl ammonium, dialkyl ammonium, trialkyl ammonium, choline, quaternary ammonium and imidazolium. Those skilled in the art will be aware of further derivatives of ammonium that carry a single positive charge that are appropriate to use in the present invention.

The nucleotide complexes may include divalent cations. The divalent cations associated with the nucleotide in the complex may comprise one or more metals selected from the list consisting of: Mg²⁺, Be²⁺, Ca²⁺, Sr²⁺, Mn²⁺ or Zn²⁺, preferably Mg²⁺ or Mn²⁺. The ratio between the divalent metal cations and the nucleotide (nucleotide ion or nucleotide ionic species) may be about 1:1 in solution, but is preferably between 0.2:1 and 2:1, optionally 0.5:1 to 1.5:1. Ratios lower than 1:1 are desirable and are preferable in DNA synthesis since ratios higher than 1:1 may lead to some infidelity in DNA synthesis. The provision of the divalent cations in relation to the nucleotide complex may therefore reduce or remove the need to add additional divalent cations to the reaction mixture. However, should further be required, these divalent cations may be provided to the enzymatic DNA synthesis in the form of any suitable salt.

Between 0.2 and 2 divalent cations may be associated with the nucleotide complex. This range includes 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 and 2 divalent cations per nucleotide complex. Those skilled in the art will appreciate the non-whole numbers represent a sharing of the divalent ion between nucleotide free acids.

The nitrogenous base may be adenine (A), guanine (G), thymine (T), cytosine (C), and uracil (U). The nitrogenous base may also be modified bases, such as 5-methylcytosine (m5C), pseudouridine (Ψ), dihydrouridine (D), inosine (I), and 7-methylguanosine (m7G). The nitrogenous base may further be an artificial base. The concentration of nucleotide complexes may include any combination of the various nitrogenous bases.

It is preferred that the five-carbon sugar is a deoxyribose, such that the nucleotide is a deoxynucleotide.

The nucleotides may be in the form of deoxynucleoside triphosphate, denoted dNTP. This is a preferred embodiment of the present invention. Suitable dNTPs may include dATP (deoxyadenosine triphosphate), dGTP (deoxyguanosine triphosphate), dTTP (deoxythymidine triphosphate), dUTP (deoxyuridine triphosphate), dCTP (deoxycytidine triphosphate), dITP (deoxyinosine triphosphate), dXTP (deoxyxanthosine triphosphate), and derivatives and modified versions thereof. It is preferred that the dNTPs comprise one or more of dATP, dGTP, dTTP or dCTP, or modified versions or derivatives thereof. It is preferred to use a mixture of dATP, dGTP, dTTP and dCTP or modified version thereof. Any suitable ratios of these dNTPs can be used, according to the needs of the reaction.

The nucleotide complexes, may already be in solution prior to mixing with the nucleotidyltransferase, or may need to be supplied as a solid for example as a powder, and dispersed in solution. The nucleotide complexes may comprise modified nucleotides. The nucleotide complexes may be provided in a mixture of one or more suitable bases, preferably, one or more of adenine (A), guanine (G), thymine (T), cytosine (C). Two, three or preferably all four nucleotides (A, G, T, and C) are used in the process to synthesise DNA. These nucleotide complexes may all be present in substantially equal amounts or more of one or two may be provided, depending on the nature of the DNA to be synthesised.

The nucleotides may all be natural nucleotides (i.e. unmodified), they may be modified nucleotides that act like natural nucleotides and are biologically active (i.e. LNA nucleotides—locked nucleic acid), they may be modified and biologically inactive or they may be a mixture of unmodified and modified nucleotides, and/or a mixture of biologically active and biologically inactive nucleotides. Each type (i.e. base) of nucleotide may be provided in one or more forms, i.e. unmodified and modified, or biologically active and biologically inactive. All of these nucleotides are capable of forming appropriate complexes.

In one aspect of the invention, the nucleotides or nucleotide complexes are present at a concentration of at least 30 mM. According to this aspect, the nucleotides or nucleotide salts may be present in the reaction mixture at a concentration of more than more than 30 mM, more than 35 mM, more than 40 mM, more than 45 mM, more than 50 mM, more than 55 mM, more than 60 mM, more than 65 mM, more than 70 mM, more than 75 mM, more than 80 mM, more than 85 mM, more than 90 mM, more than 95 mM, or more than 100 mM. Such concentrations are given as the concentration of nucleotide complex at the initiation or start of the process. The concentration is given after the addition of the nucleotide/nucleotide complexes, wherein the addition may be to the reaction mixture. The nucleotide complex may be any appropriate mixture of nucleotide complexes, with varying nitrogenous bases. The concentration applies to the sum total of nucleotide complexes present in at the start of the process, whatever their composition. Thus, for example, a 30 mM concentration of nucleotide salts may be any mixture of dCTP, dATP, dGTP and dTTP counter-ioned with appropriate monovalent and divalent cations.

It will be understood that nucleotides supplied as complexes may dissociate in water and other solvents to form an anionic nucleotide entity (nucleotide ion, nucleotide ionic species) and the associated cations.

It is a preferred part of any aspect of the present invention that the nucleotide complex is formed by a mixture of counter-ions The nucleotide complexes used in the processes of the inventions include a mixture of different cation species; notably at least one species of divalent cation and at least one species of monovalent cation. Preferably the ratio of monovalent cation:nucleotide is between 0.2:1 and 2.5:1, optionally between 0.5:1 to 2:1. Preferably the ratio of divalent cation:nucleotide is between 0.2:1 and 2:1, optionally between 0.5:1 to 1.5:1, preferably 0.5:1 up to 1:1. The enzymatic DNA synthesis may be maintained under conditions promoting synthesis of DNA, and this will depend upon the particular method selected.

Amplification of a template via strand displacement is preferred. Preferably, the conditions promote amplification of said template by displacement of replicated strands through strand displacement replication of another strand. The conditions comprise use of any temperature allowing for amplification of DNA, commonly in the range of 20 to 90 degrees centigrade. A preferred temperature range may be about 20 to about 40 or about 25 to about 35 degrees centigrade. A preferred temperature for LAMP amplification is about 50 to about 70 degrees centigrade.

Typically, an appropriate temperature for enzymatic DNA synthesis is selected based on the temperature at which a specific polymerase has optimal activity. This information is commonly available and forms part of the general knowledge of the skilled person. For example, where phi29 DNA polymerase is used, a suitable temperature range would be about 25 to about 35 degrees centigrade, preferably about 30 degrees centigrade. However, a thermostable phi29 may operate at a higher constant temperature. The skilled person would routinely be able to identify a suitable temperature for efficient amplification according to the processes of the invention. For example, a process could be carried out at a range of temperatures, and yields of amplified DNA could be monitored to identify an optimal temperature range for a given polymerase. The amplification may be carried out at a constant temperature, and it is preferred that the process is isothermal. Since strand displacement amplification is preferred there is no requirement to alter the temperature to separate DNA strands. Thus, the process may be an isothermal process.

Other conditions promoting DNA synthesis are conventionally thought to comprise the presence of suitable buffering agents/pH and other factors which are required for enzyme performance or stability. Suitable conventional conditions include any conditions used to provide for activity of polymerase enzymes known in the art.

For example, the pH of the reaction mixture may be within the range of 3 to 10, preferably 5 to 8 or about 7, such as about 7.5. pH may be maintained in this range by use of one or more buffering agents (also called pH buffering agents). The function of a buffering agent is to prevent a change in pH. Such buffers (buffering agents) include, but are not restricted to MES, Bis-Tris, ADA, ACES, PIPES, MOBS, MOPS, MOPSO, Bis-Tris Propane, BES, TES, HEPES, DIPSO, TAPSO, Trizma, HEPPSO, POPSO, TEA, EPPS, Tricine, Gly-Gly, Bicine, HEPBS, TAPS, AMPD, TABS, AMPSO, CHES, CAPSO, AMP, CAPS, CABS, phosphate, citric acid-sodium hydrogen phosphate, citric acid-sodium citrate, sodium acetate-acetic acid, imidazole and sodium carbonate-sodium bicarbonate. Preferred are buffering agents that do not provide further cations to the reaction mixture, nor complex with metal cations present in the reaction mixture as discussed previously.

A buffer is generally defined by a mixture of reaction components. Usually included is a buffering agent to maintain a stable pH; one or more additional salts composed of a cationic and anionic species i.e. sodium chloride, potassium chloride; and/or detergents such as Triton-X-100 to ensure optimal activity or stability of the enzymes. A minimal buffer is composed of only a buffering reagent with no additional salts or detergents provided, with the proviso that small amounts of cationic species may be present for DNA synthesis in which chemical denaturation is required. Surprisingly, using higher concentrations of nucleotide salts in the processes of the invention permits the use of these minimal buffers.

A “no buffer” system lacks a provided or defined pH buffering agent in the mixture of reaction components and lacks additional salts or detergents. This “no added buffering agent” system contains only the reaction components required for the DNA synthesis alone, and contains cationic species provided for chemical denaturation only (if required). Thus, in this system, there are no additional ions added beyond those that serve a specific purpose in the DNA synthesis reaction. The counter-ions provided with the nucleotides (as a complex) serve to stabilise the nucleotide prior to use in the process.

While the application of heat (exposure to 95° C. for several minutes) is used to denature double stranded DNA other approaches may be used which are more suitable for DNA synthesis. Double stranded DNA can be readily denatured by exposure to a high or low pH environment or where cations are absent or present in very low concentrations, such as in deionized water. The polymerase requires the binding of a short oligonucleotide primer sequence to a single stranded region of the DNA template to initiate its replication. The stability of this interaction and therefore the efficiency of DNA synthesis may particularly be influenced by the concentration of metal cations and particularly divalent cations such as magnesium (Mg²⁺) ions which may be seen as an integral part of the process.

The enzymatic DNA synthesis may also require the presence of additional divalent metal ions, namely divalent cations that are supplied externally to the nucleotide complex. The process may comprise the use of salts of divalent metal ions: magnesium (Mg²⁺), manganese (Mn²⁺), calcium (Ca²⁺), beryllium (Be²⁺), zinc (Zn²⁺) and strontium (Sr²⁺). The most often used divalent ions in DNA synthesis is magnesium or manganese, since these act as a cofactor in DNA synthesis. Any suitable anion may be utilised in such salts, whilst being mindful that the choice of anion can have an effect on the pH of the reaction mixture, and should be suitably accounted for.

Detergents may also be included in the reaction mixture in certain aspects. Examples of suitable detergents include Triton X-100™, Tween 20™ and derivatives of either thereof. Stabilising agents may also be included in the reaction mixture. Any suitable stabilising agent may be used, in particular, bovine serum albumin (BSA) and other stabilising proteins. Reaction conditions may also be improved by adding agents that relax DNA and make template denaturation easier. Such agents include, for example, dimethyl sulphoxide (DMSO), formamide, glycerol and betaine. DNA condensing agents may also be included in the reaction mixture. Such agents include, for example, polyethylene glycol or cationic lipid or cationic polymers.

However, in certain embodiments, these components may be reduced or removed from the reaction mixture, for example in the minimal or no added buffering agent systems.

It should be understood that the skilled person is able to modify and optimise synthesis conditions for the processes of the invention using these additional components and conditions on the basis of their general knowledge. Likewise the specific concentrations of particular agents may be selected on the basis of previous examples in the art and further optimised on the basis of general knowledge.

As an example, a suitable reaction buffer used in RCA-based methods in the art is 50 mM Tris HCl, pH 7.5, 10 mM MgCl₂, 20 mM (NH₄)₂SO₄, 5% glycerol, 0.2 mM BSA, 1 mM dNTPs. A preferred reaction buffer used in the RCA amplification is usually 30 mM Tris-HCl pH 7.9, 30 mM KCl, 7.5 mM MgCl₂, 10 mM (NH₄)₂SO₄, 4 mM DTT, 2 mM dNTPs. This buffer is particularly suitable for use with Phi29 DNA polymerase when conventional nucleotides are purchased.

A suitable reaction buffer for use with the nucleotide complexes of the invention is 60 mM Tris pH 8.0. A further suitable reaction buffer is 30 mM Tris pH 8.0. Alternative conditions include 30 mM Tris HCl, pH 7.9, 5 mM (NH₄)₂SO₄, and 30 mM KCl. Under certain circumstances, the enzymatic DNA synthesis may be conducted in water (“no added buffering agent”).

The enzymatic DNA synthesis may also comprise the use of one or more additional proteins. The template may be amplified in the presence of at least one pyrophosphatase, such as Yeast Inorganic pyrophosphatase. Two, three, four, five or more different pyrophosphatases may be used. These enzymes are able to degrade pyrophosphate generated by the polymerase from dNTPs during strand replication. Build-up of pyrophosphate in the reaction can cause inhibition of DNA polymerases and reduce speed and efficiency of DNA amplification. Pyrophosphatases can break down pyrophosphate into non-inhibitory phosphate. An example of a suitable pyrophosphatase for use in the processes of the present invention is Saccharomyces cerevisiae pyrophosphatase, available commercially from New England Biolabs, Inc.

Any single-stranded binding protein (SSBP) may be used in the processes of the invention, to stabilise single-stranded DNA. SSBPs are essential components of living cells and participate in all processes that involve ssDNA, such as DNA replication, repair and recombination. In these processes, SSBPs bind to transiently formed ssDNA and may help stabilise ssDNA structure. An example of a suitable SSBP for use in the processes of the present invention is T4 gene 32 protein, available commercially from New England Biolabs, Inc.

The yield of the reaction relates to the amount of DNA synthesised. The expected yield from a process according to the present invention may exceed 3 g/l. It is preferred that the amount of DNA synthesised is greater than 3, 4, 5, 6, 7, 8, 9, 10, 11, 12, 13, 14, 15, 16, 17, 18, 19, 20, 21, 22, 23, 24, 25, 26, 27, 28, 29 or 30 g/l or more. A preferred amount of DNA synthesised is 5 g/l. 30 mM nucleotide complex is capable of generating 9.74 g/l DNA. The present invention improves the yield possible from enzymatic synthesis of DNA. It is an object of the present invention to improve the yield of a cell-free enzymatic DNA synthesis process, such that DNA can be synthesised on a large scale in a cost-effective way. The present invention allows the manufacture/synthesis of DNA economically on an industrial scale using an enzymatic process catalysed by a DNA synthesis enzyme or polymerase. The present process allows the efficient incorporation of nucleotides into the DNA product. It is thought that the processes of the invention will allow reaction mixtures to be scaled up into several litres, including tens of litres. The improved yield, productivity or processivity may be compared to an identical reaction mixture where all of the nucleotides are supplied as conventional salts with monovalent cation counter-ions (generally lithium or sodium).

In one embodiment, the present invention relates to a process for enhancing the synthesis of DNA. This enhancement may be compared to an identical reaction mixture, with the exception that all of the nucleotides complexes used are exclusively monovalent cation counter-ions, or a mixture thereof.

In one aspect, the invention provides a cell-free process for synthesising DNA comprising contacting a DNA template with at least one nucleotidyltransferase in the presence of one or more nucleotide complexes, wherein each of said nucleotide complexes are associated with between 0.2 and 2 divalent cations and between 0.2 and 2.5 monovalent cations.

It is preferred that the concentration of nucleotides referred to herein is the starting concentration of nucleotides at the start of the process, the initial concentration when the reaction mixture is formed.

The invention may also relate to a cell-free process for synthesising DNA comprising contacting a DNA template with at least one nucleotidyltransferase in the presence of one or more nucleotide complexes in a concentration of over 30 mM. The invention provides a cell-free process for the enzymatic synthesis of DNA comprising the use of nucleotides supplied as complexes, wherein said each of said complexes are a nucleotide associated with between 0.2 and 2 divalent cations and between 0.2 and 2.5 monovalent cations, preferably wherein the nucleotide complexes are obtained, supplied or are present in a concentration greater than 30 mM.

The invention further provides an enzymatic DNA synthesis which is performed under conditions of reduced or even no further additionally supplied divalent cations, preferably magnesium, comprising the use of nucleotide complexes, wherein each of said complexes comprise a nucleotide associated with between 0.2 and 2 divalent cations and between 0.2 and 2.5 monovalent cations. The provision of the divalent cation in the nucleotide complex avoids the further use of divalent cation salts in the process. However, in certain circumstances, the amount of divalent cation salts such as magnesium, are reduced using the complexes of the invention.

The invention will now be described with reference to several non-limiting examples.

### Example 1

The Effects of Mono-Cation Concentration in Nucleotide Complexes on DNA Synthesis

Materials and Methods

Reagents

The following reagents supplied were used in the presented examples:
Solution 1-100 mM dATP:4Na⁺
Solution 2-100 mM dCTP:4Na⁺
Solution 3-100 mM dGTP:4Na⁺
Solution 4-100 mM dTTP: 4Na⁺
Solution 5-100 mM dATP: 4NH₄⁺
Solution 6-100 mM dCTP: 4NH₄⁺
Solution 7-100 mM dGTP: 4NH₄⁺
Solution 8-100 mM dTTP: 4 NH₄⁺
Solution 9-25 mM dATP:1.6 Mg²⁺
Solution 10-61 mM dCTP:2.0 Mg²⁺
Solution 11-34 mM dGTP: 1.8 Mg²⁺
Solution 12-91 mM dTTP: 2.5 Mg²⁺
Phi29 DNA polymerase, stock concentration 5.6 g/L (produced in-house)
Thermostable pyrophosphatase, stock concentration 2000 U/ml (Enzymatics)
DNA primer, stock concentration 5 mM (Oligofactory)
Plasmid template: ProTLx-K B5X4 LUX 15-0-15-10-15 AT-STEM, stock concentration 0.832 g/L (produced in house)
Nuclease free water (Sigma Aldrich)
1M NaOH (Sigma Aldrich)
Magnesium Acetate, stock concentration 1M (Sigma Aldrich)
Tris-base (Thermo Fisher Scientific)
Tris-HCl (Sigma Aldrich)
NaCl (Sigma Aldrich)
EDTA, stock concentration 0.5 M (Sigma Aldrich)
PEG 8000 (AppliChem)

Preparation of dNTP Mixes

For both the sodium complex (dNTP:4Na⁺) and ammonium complex (dNTP: 4NH₄⁺) the individual dNTPS (solutions 1, 2, 3, 4 and 5, 6, 7, 8 respectively) were mixed 1:1:1:1 to produce a stock concentration of 100 mM dNTP mixture. The dNTP mixes were stored at −20° C. until ready for use.

For sodium/magnesium complexes (dNTPs:Na⁺/Mg²⁺) and ammonium/magnesium complexes (dNTPs:NH₄⁺/Mg²⁺) dNTPS were mixed as follows to provide an equal molar amount of each mononucleotide

TABLE 1
Table 1: Volumes of dNTPS used to
form monovalent:divalent dNTP mixes

| Magnesium | | | Ammonium or Sodium | | |
|---|---|---|---|---|---|
| dNTP complex | | | dNTP complex | | |
| | | Vol- | | | Vol- |
| | [dXTP] | ume | | [dXTP] | ume |
| dNTP | mM | (μl) | dNTP | mM | (μl) |
| dATP (soln 9) | 25 | 1820 | dATP (soln 1 or 5) | 100 | 455 |
| dCTP (soln 10) | 61 | 745 | dCTP (soln 2 or 6) | 100 | 455 |
| dGTP (soln 11) | 34 | 1338.2 | dGTP (soln 3 or 7) | 100 | 455 |
| dTTP (soln 12) | 91 | 500 | dTTP (soln 4 or 8) | 100 | 455 |

Mixing the solutions in Table 1 in the volumes indicated yields a stock concentration of 58 mM dNTPs in a ratio of 1:1 with Mg²⁺ and 1:2 with either Na⁺ or NH₄⁺. To achieve a final stock concentration of 100 mM dNTP mix, the final volume of 6223 μl was reduced to 3640 μl using an Eppendorf SpeedVac Concentrator Plus operating at a temperature of 60° C.

Due to low solubility of dNTP:Mg²⁺, it was not possible to perform the following experiments using magnesium complexed dNTPs alone.

DNA Amplification Reaction Setup

Reactions were set up at 500 μl scale as follows: A denaturation mix was prepared, and left at room temperature while the reaction mix was assembled. These were then mixed, and the DNA polymerase and pyrophosphatase added. DNA amplification experiments were then performed at a range of dNTP concentrations in a reaction buffer containing 60 mM Tris, pH 8.0. For sodium and ammonium complexed dNTPS (i.e. dNTP:4Na⁺ and dNTP:4NH₄⁺) an equimolar amount of magnesium acetate was added to the reaction mix while for dNTPs complexed with sodium and magnesium or ammonium and magnesium, as described above, no additional magnesium was supplied to the reaction mix. Table 2 shows the experimental protocol for the DNA synthesis reactions.

The experiments were carried out to determine if reducing monovalent counterions on the dNTPS would result in higher dNTP usage. Reactions were grown for 168 hr (1 week) at a temperature of 30° C. followed by processing and quantification.

TABLE 2
DNA synthesis by Rolling Circle Amplification (RCA) Reaction components
for examining the effects mono- and di-cation dNTP complexes.

| | | Stock | | | | Final reaction | |
|---|---|---|---|---|---|---|---|
| | Reagent | concentration | | Volume | | concentration | |
| Denaturation | Plasmid Template | 0.832 | g/l | 1.2 | μl | 2 | ng/μl |
| mix | NaOH | 1M | | 2.5 | μl | 5 | mM |
| | DNA primer | 5 | mM | 5 | μl | 50 | μM |
| | H₂O | | | 18.8 | μl | to 25 μl | |
| Reaction | Tris pH 8.0 | 1M | | 30 | ul | 60 | mM |
| mix | dNTP salts | 100 | mM | Variable | | Variable - as shown | |
| | H₂O | | | Variable | | to 500 μl total | |
| Enzyme 1 | Phi29 DNA | 5.6 | g/l | 0.25 | μl | | |
| | polymerase | | | | | | |
| Enzyme 2 | Pyrophosphatase | 2000 | U/ml | 0.25 | μl | 0.4 | U |

Sample Processing Procedure

222 mM EDTA was added after 168 hrs of RCA to a volume of 900 μl. 300 μl of water was added followed by mixing on “an end over end” rotary mixer over 4 hrs. 400 μl of 5M NaCl was added followed by 400 μl of 50% PEG 8000 (w/v). The reaction tubes were shaken vigorously for 15 minutes followed by further rotary mixing for 4 hrs. Precipitated DNA was recovered by centrifugation at 13,000 rpm in a bench-top centrifuge for 10 minutes. The supernatants were carefully decanted, and the pellets were resuspending in 9000 μl water overnight on an end over end mixer. Reaction DNA concentrations were quantified from UV absorption measurements on a nanodrop spectrophotometer. Data is corrected for the 18× fold increase in reaction volume and concentrations are expressed in g/l of original volume vs dNTP concentrations used.

Results

TABLE 3
DNA yields with different dNTP cation complexes at different
concentrations. Peak yields are highlighted in bold

| | Raw DNA yield (g/l) | | | |
|---|---|---|---|---|
| | dNTPS: | dNTP: | dNTP: | dNTP: |
| | 4Na⁺ | 2Na•Mg²⁺ | 4NH₄⁺ | 2NH₄⁺•Mg²⁺ |
| 10 mM | 2.863 | 1.900 | Not performed | Not performed |
| 20 mM | 5.436 | 3.649 | 5.870 | 4.165 |
| 30 mM | 7.611 | 4.938 | 9.57 | 8.402 |
| 40 mM | 5.332 | 6.606 | 9.867 | 9.119 |
| 50 mM | 1.214 | 7.817 | 3.997 | 9.569 |
| 60 mM | 0.918 | 9.218 | 1.781 | 12.137 |
| 70 mM | 0.926 | 10.164 | 1.597 | 12.042 |
| 80 mM | 1.394 | 11.106 | 1.798 | 13.426 |

TABLE 4
DNA Efficiency with different dNTP cation
complexes at different concentrations.

| | Incorporation efficiency (%) | | | |
|---|---|---|---|---|
| | dNTPS: | dNTP: | dNTP: | dNTP: |
| | 4Na⁺ | 2Na•Mg²⁺ | 4NH₄⁺ | 2NH₄⁺•Mg²⁺ |
| 10 mM | 88.1 | 58.5 | Not performed | Not performed |
| 20 mM | 83.6 | 56.1 | 90.3 | 64.1 |
| 30 mM | 78.1 | 50.6 | 98.2 | 86.2 |
| 40 mM | 41.0 | 50.8 | 75.9 | 70.1 |
| 50 mM | 7.5 | 48.1 | 24.6 | 58.9 |
| 60 mM | 4.7 | 47.3 | 9.1 | 62.2 |
| 70 mM | 4.1 | 44.7 | 7.0 | 52.9 |
| 80 mM | 5.4 | 42.7 | 6.9 | 51.6 |

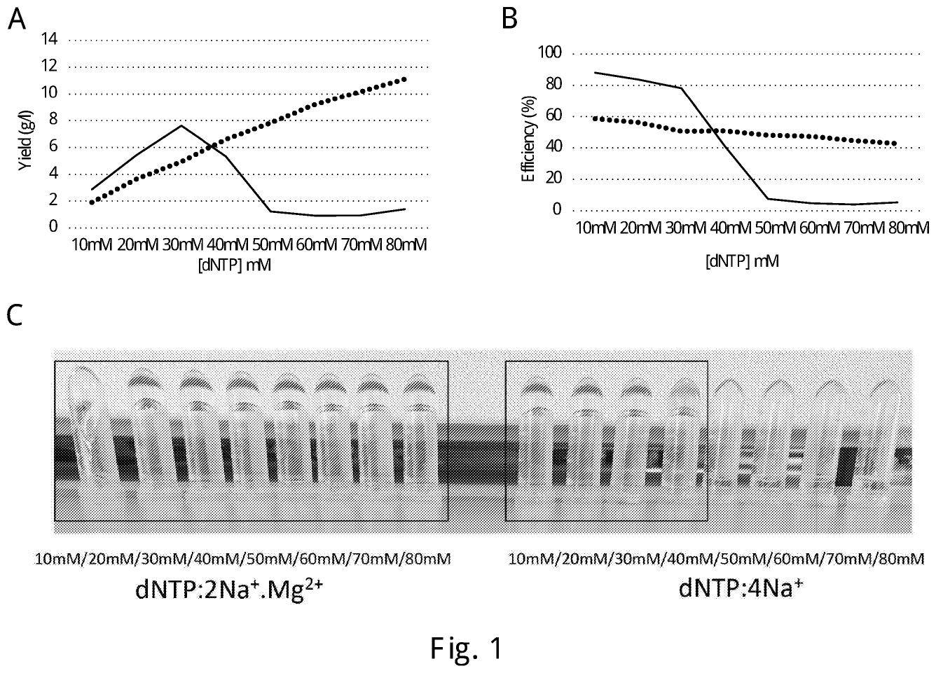

The data in Tables 3 (represented graphically and also physically in FIG. 1) demonstrate that on reducing the concentrations of monovalent sodium in dNTP complexes by the addition of magnesium cations there is an increase in raw DNA produced. By using dNTPs counterioned with monovalent/divalent mixture i.e. dNTP:2Na⁺.Mg²⁺ the highest level of dNTP usage increases from 30 mM with standard dNTP:4Na⁺ to at least 80 mM and the DNA yield from a peak of 7.611 g/l to 11.106 g/l respectively. Although the efficiency of conversion of dNTPS into DNA was lower for the dNTP:2Na⁺.Mg²⁺ mix at lower concentrations compared to dNTP:4Na⁺, decrease in overall efficiency was lower for dNTP:2Na⁺.Mg²⁺ over the range of experimental conditions (Table 4). DNA viscosity was also viewed after 168 hrs of growth and it can be seen that dNTP:2Na⁺.Mg²⁺ produces viscous DNA material up to 80 mM while dNTP:4Na⁺ peaks at 40 mM, with the higher concentrations of dNTPS producing low viscosity material.

Compared to the corresponding sodium complexed dNTPs, the use of ammonium dNTP complexes (dNTP:4NH₄⁺) results in peak production shifting higher to 40 mM dNTP concentrations producing raw yields of 9.867 g/l, almost two fold higher than the corresponding sodium counterbalanced dNTP (dNTP:4Na⁺).

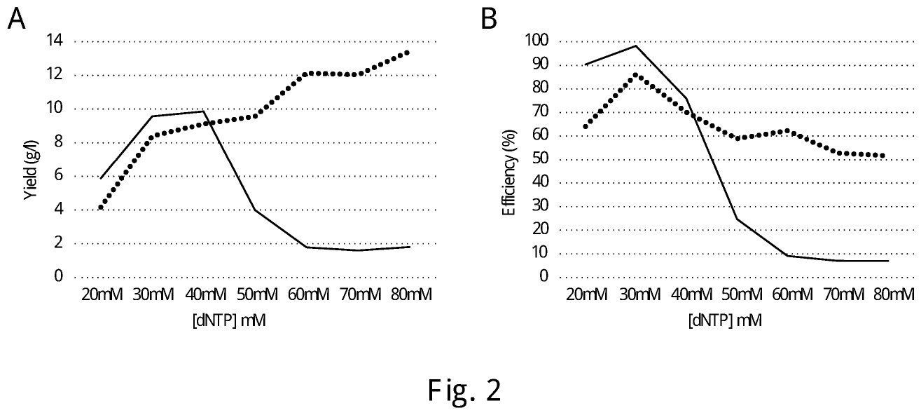

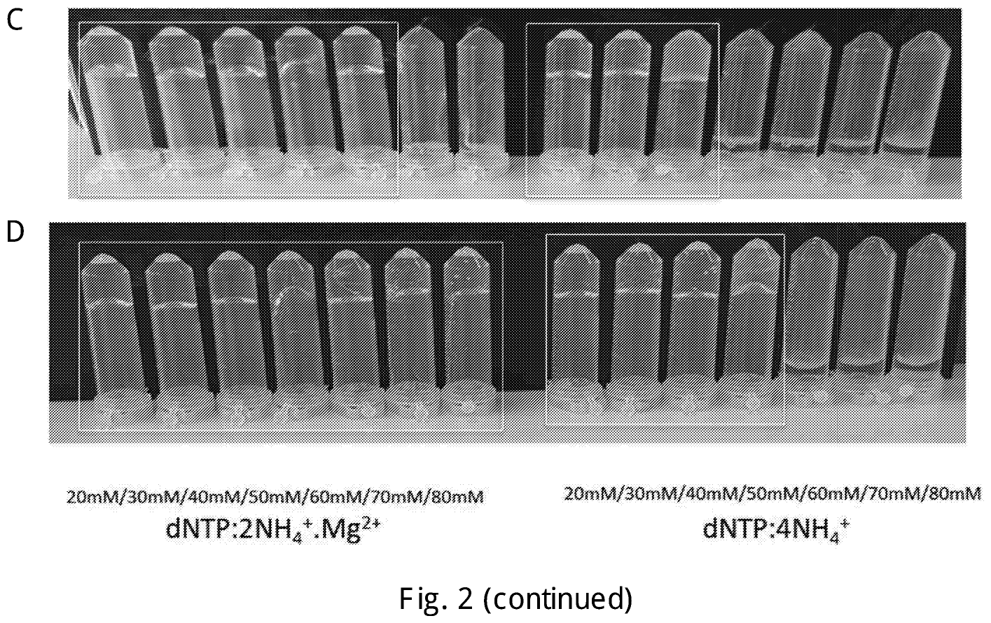

However, the dNTP:2NH₄⁺.Mg²⁺ complex has further increased usage to 80 mM and has resulted in a yield increase to at least 13.426 g/l. Also, as can be seem from FIGS. 2C and 2D, the use of dNTP:2NH₄⁺.Mg²⁺ complex also results in an increased rate of DNA production as seen by DNA viscosity. After 18 hrs dNTP: 4NH₄⁺ reactions have produced highly viscous material up to a concentration of 30 mM while dNTP:2NH₄⁺.Mg²⁺ reactions have reached 60 mM. After 106 hrs all concentrations of dNTP:2NH₄⁺.Mg²⁺ have produced highly viscous material, while dNTP: 4NH₄⁺ have only produced highly viscous materials up to 40 mM. No further increase in viscosity was observed after this time point

The figures demonstrating the viscosity of the reaction mixture once DNA has been synthesised are a very visual representation of the amount of DNA the process is able to synthesise. Once the DNA synthesis has taken place, the tubes have been inverted. Where no DNA or very little DNA is synthesised, the reaction mixture does not become viscous, and the reaction mixture collects at the cap of the tube. Where a sufficient amount of DNA is produced, the reaction mixture becomes very viscous, allowing the tube to be inverted and for the reaction mixture to remain in place in the tube. The more DNA, the more viscous the reaction mixture and the greater the retention in the tube. It can be seen that slightly less viscous products start to slip down the inside of the tubes once inverted.

### Example 2

Effect of Different Monovalent Cations in Nucleotide Complexes with Magnesium on DNA Yield at a Range of Concentrations.

Materials and Methods

Reagents

The following reagents supplied were used in the presented examples:
Solution 1-100 mM dATP: 4 K⁺
Solution 2-100 mM dCTP: 4 K⁺
Solution 3-100 mM dGTP: 4 K⁺
Solution 4-100 mM dTTP: 4 K⁺
Solution 5-100 mM dATP: 4 Cs⁺
Solution 6-100 mM dCTP: 4 Cs⁺
Solution 7-100 mM dGTP: 4 Cs⁺
Solution 8-100 mM dTTP: 4 Cs⁺
Solution 9-200 mM dATP: 4 NH₄⁺
Solution 10-200 mM dCTP: 4 NH₄⁺
Solution 11-200 mM dGTP: 4 NH₄⁺
Solution 12-200 mM dTTP: 4 NH₄⁺
Solution 13-66 mM dATP: 2 Mg²⁺
Solution 14-59 mM dCTP: 2 Mg²⁺
Solution 15-64 mM dGTP: 2 Mg²⁺
Solution 16-74 mM dTTP: 2 Mg²⁺
Phi29 DNA polymerase, stock concentration 5.6 g/L (produced in-house)
Thermostable pyrophosphatase, stock concentration 2000 U/ml (Enzymatics)
DNA primer, stock concentration 5 mM (Oligofactory)
Plasmid template: ProTLx-K B5X4 LUX 15-0-15-10-15 AT-STEM, stock concentration 0.832 g/L (produced in house)
Nuclease free water (Sigma Aldrich)
1M NaOH (Sigma Aldrich)
PEG 8000 (Applichem)
Tris-Base (Thermo Fisher Scientific)
Tris-HCl (Sigma Aldrich)
NaCl (Sigma Aldrich)

Preparation of dNTP Mixes

For the potassium (dATP: 4 K⁺), caesium (dATP: 4 Cs⁺) and ammonium (dATP: 4 NH₄⁺) complexes the individual dNTPs (sol 1 to 12) were mixed 1:1:1:1 to produce a stock concentration of 100 mM dNTP mixture for potassium and caesium, while a 200 mM dNTP mixture for ammonium. The mixes were stored at −20° C.

For the magnesium mixed complexes (i.e. dNTPs: K⁺/Mg²⁺ or Cs⁺/Mg²⁺ or NH₄⁺/Mg²⁺), dNTPs were mixed in such a way to provide an equimolar amount of each specific nucleotide (i.e. dATP, dCTP, dGTP & dTTP).

TABLE 5

| Magnesium | | | Potassium or Caesium | | |
|---|---|---|---|---|---|
| dNTP Complex | | | dNTP Complex | | |
| | [dXTP] | Volume | | [dXTP] | Volume |
| dNTP | mM | (μl) | dNTP | mM | (μl) |
| dATP (sol 13) | 66 | 1515 | dATP (sol 1 or 5) | 100 | 1000 |
| dCTP (sol 14) | 59 | 1695 | dCTP (sol 2 or 6) | 100 | 1000 |
| dGTP (sol 15) | 64 | 1563 | dGTP (sol 3 or 7) | 100 | 1000 |
| dTTP (sol 16) | 74 | 1351 | dTTP (sol 4 or 8) | 100 | 1000 |

TABLE 6

| Magnesium dNTP Complex | | | Ammonium dNTP Complex | | |
|---|---|---|---|---|---|
| | [dXTP] | Volume | | [dXTP] | Volume |
| dNTP | mM | (μl) | dNTP | mM | (μl) |
| dATP (sol 13) | 66 | 1515 | dATP (sol 9) | 200 | 500 |
| dCTP (sol 14) | 59 | 1695 | dCTP (sol 10) | 200 | 500 |
| dGTP (sol 15) | 64 | 1563 | dGTP (sol 11) | 200 | 500 |
| dTTP (sol 16) | 74 | 1351 | dTTP (sol 12) | 200 | 500 |

Magnesium nucleotides were mixed in such a way to provide equimolar amounts of each nucleotide. The volumes correspond to 100 mM of each nucleotide such that the final volume was 4000 μL.

Far caesium and potassium nucleotides, the final volume of 6124 μL was reduced to powder (i.e 0 μL) in a Speedvac at 60° C. The powder was resuspended by using the caesium or potassium pre-mixed nucleotides as detailed in Table 5, to a final volume of 4000 μL resulting in 200 mM K⁺/Mg²⁺ or Cs⁺/Mg²⁺ dNTPs.

For ammonium nucleotide, the final volume of 6124 μL was reduced to powder (i.e 0μL) in a Speedvac at 60° C. The powder was resuspended by using the ammonium pre-mixed nucleotides as detailed in Table 6 with an additional 2000 μl H₂O to a final volume of 4000 μl resulting in 200 mM NH₄⁺/Mg²⁺ dNTPs.

DNA Amplification Reaction Setup

Reactions were set up at 500 μl scale as follows: A denaturation mix was prepared and left at room temperature for 15 minutes while the reaction mix was assembled. These were then mixed, and the DNA polymerase and pyrophosphatase added. DNA amplification experiments were then performed at a range of dNTP concentrations with 30 mM pH 8.0 Tris buffer added. Reactions were split into 5 100 μl aliquots and stopped after 48, 72, 96, 120 and 144 hours. After stopping the samples were immediately processed as detailed below.

For mono-counterion complexed dNTPs (for example potassium dNTPs) an equimolar amount of magnesium chloride was added to the reaction mix. While for dNTPs complexed with both monovalent counterions (i.e. NH₄⁺, K⁺, Cs⁺) and magnesium, as described above, no additional magnesium was supplied to the reaction mix. Table 5 shows the experimental protocol for the mono-complexed dNTPs (NH₄⁺, K⁺& Cs⁺) reaction setup while Table 6 shows the reaction setup for the mixed dNTP complexes dNTPs (NH₄⁺/Mg²⁺, K⁺/Mg²⁺& Cs⁺/Mg²⁺)

The experiments were carried out to determine if reducing monovalent counterions on the dNTPs would result in higher dNTP usage. Reactions were grown for the specified time period at a temperature of 30° C. followed by processing and quantification.

TABLE 7
DNA synthesis by Rolling Circle Amplification (RCA) Reaction components
for examining the effects mono- and di-cation dNTP complexes.

| | | Stock | | | | Final reaction | |
|---|---|---|---|---|---|---|---|
| | Reagent | concentration | | Volume | | concentration | |
| Denaturation | Plasmid Template | 0.832 | g/l | 1.2 | μL | 2 | ng/μL |
| mix | NaOH | 1M | | 2.5 | μL | 5 | mM |
| | DNA primer | 5 | mM | 5 | μL | 50 | μM |
| | H₂O | | | 18.8 | μL | to 25 μL | |
| Reaction mix | Tris pH 8.0 | 1M | | 15 | μL | 30 | mM |
| | Mono dNTP salts | 100 | mM | Variable | | Variable - as shown | |
| | MgCl₂ | 2000 | mM | Variable | | Equimolar levels to dNTP | |
| | | | | | | molar concentrations | |
| | H₂O | | | Variable | | to 500 μL total | |
| Enzyme 1 | Phi29 | 5.6 | g/L | 0.25 | μL | | |
| | DNA polymerase | | | | | | |
| Enzyme 2 | Pyrophosphatase | 2000 | U/mL | 0.5 | μL | 1 | U |

TABLE 8
DNA synthesis by Rolling Circle Amplification (RCA. Reaction components
for examining the effects mono- and di-cation dNTP complexes.

| | | Stock | | | | Final reaction | |
|---|---|---|---|---|---|---|---|
| | Reagent | concentration | | Volume | | concentration | |
| Denaturation | Plasmid Template | 0.832 | g/l | 1.2 | μL | 2 | ng/μL |
| mix | NaOH | 1M | | 2.5 | μL | 5 | mM |
| | DNA primer | 5 | mM | 5 | μL | 50 | μM |
| | H₂O | | | 18.8 | μL | to 25 μL | |
| Reaction mix | Tris pH 8.0 | 1M | | 15 | μL | 30 | mM |
| | Mixed dNTP salts | 200 | mM | Variable | | Variable - as shown | |
| | H₂O | | | Variable | | to 500 μL total | |
| Enzyme 1 | Phi29 | 5.6 | g/L | 0.25 | μL | | |
| | DNA polymerase | | | | | | |
| Enzyme 2 | Pyrophosphatase | 2000 | U/mL | 0.5 | μL | 1 | U |

Sample Processing Procedure

To each aliquot 900 μl of water was added for dilution and then 200 μl 5M NaCl and 500 μl of 25% PEG 8000 was added, solutions mixed by shaken vigorously for 15 minutes followed by further rotary mixing for 1 hour. The DNA was pelleted by centrifugation in a microcentrifuge (13,000 rpm, 30 minutes). The supernatants were carefully decanted, and the pellets were resuspended in 1000 μl water by vigorous shaking and air-displacement pipetting. Reaction DNA concentrations were quantified from UV absorption measurements on a nanodrop spectrophotometer at the end of the day and then left rotating overnight. Samples were re-checked in the morning and non reported any differences from previous day.

Data is corrected for the 10× fold increase in reaction volume and concentrations are expressed in g/l of original volume vs dNTP concentrations used. However, the high DNA yields may be underestimations due to the high difficulty in fully resuspending and solubilising very thick viscous DNA gels.

Results

TABLE 9
DNA yields with different dNTP cation complexes at different concentrations.

| | | Raw DNA Yield (g/l) | | | | | |
|---|---|---|---|---|---|---|---|
| | | dNTP: | dNTP: | dNTP: | dNTP: | dNTP: | dNTP: |
| | | 4K⁺ | 2K⁺•Mg²⁺ | 4NH₄⁺ | 2NH₄⁺•Mg²⁺ | 4Cs⁺ | 2Cs⁺•Mg²⁺ |
| 5 | mM | 1.65 | 1.11 | 1.24 | 1.10 | 1.18 | 1.12 |
| 10 | mM | 3.09 | 2.16 | 2.64 | 2.54 | 2.95 | 2.52 |
| 20 | mM | 5.52 | 4.65 | 5.18 | 3.98 | 5.69 | 3.88 |
| 30 | mM | 7.76 | 7.40 | 7.39 | 5.88 | 7.55 | 5.45 |
| 40 | mM | 1.41 | 7.60 | 9.78 | 9.26 | 7.65 | 7.68 |
| 80 | mM | 1.76 | 11.12 | 1.62 | 13.35 | 1.90 | 9.40 |
| 100 | mM | Not | 2.31 | 1.99 | 15.89 | Not | 7.75 |
| | | Performed | | | | Performed | |
| 120 | mM | Not | 2.00 | 2.20 | 6.98 | Not | 2.15 |
| | | Performed | | | | Performed | |

TABLE 10
DNA Efficiency with different dNTP cation
complexes at different concentrations.

| | | Incorporation Efficiency (%) | | | | | |
|---|---|---|---|---|---|---|---|
| | | dNTP: | dNTP: | dNTP: | dNTP: | dNTP: | dNTP: |
| | | 4K⁺ | 2K⁺•Mg²⁺ | 4NH₄⁺ | 2NH₄⁺•Mg²⁺ | 4Cs⁺ | 2Cs⁺•Mg²⁺ |
| 5 | mM | 101.5% | 68.3% | 76.3% | 67.7% | 72.6% | 68.9% |
| 10 | mM | 95.1% | 66.5% | 81.2% | 78.2% | 90.8% | 77.5% |
| 20 | mM | 84.9% | 71.5% | 79.7% | 61.2% | 87.5% | 59.7% |
| 30 | mM | 79.6% | 75.9% | 75.8% | 60.3% | 77.4% | 55.9% |
| 40 | mM | 10.8% | 58.5% | 75.2% | 71.2% | 58.8% | 59.1% |
| 80 | mM | 6.8% | 42.8% | 6.2% | 51.3% | 7.3% | 36.2% |
| 100 | mM | Not | 7.1% | 6.1% | 48.9% | Not | 23.8% |
| | | Performed | | | | Performed | |
| 120 | mM | Not | 5.1% | 5.6% | 17.9% | Not | 5.5% |
| | | Performed | | | | Performed | |

The data in Table 9 demonstrates that by reducing the concentrations of monovalent counterions in dNTP complexes by the addition of magnesium cations, there is an increase in raw DNA produced. By using dNTPs with mixed counterions of a monovalent/divalent mixture i.e dNTP:2K⁺. Mg²⁺ the level of dNTP usage increases from 30 mM with standard dNTP:4K⁺ to at least 80 mM and the DNA yield from a peak of 7.76 g/l to 11.12 g/l respectively (Table 9).

Although the efficiency of conversion of dNTPs into DNA was lower for the dNTP:2K⁺.Mg²⁺ mix at lower concentrations compared to dNTP:4K⁺ decrease in overall efficiency was lower for dNTP:2K⁺.Mg²⁺ over the range of experimental conditions (Table 10).

By using dNTPs with mixed counterions of a monovalent/divalent mixture for ammonium dNTP:2NH₄⁺.Mg²⁺ the level of dNTP usage increases from 40 mM with standard dNTP:4NH₄⁺ to at least 100 mM and the DNA yield from a peak of 9.78 g/l to 15.89 g/l respectively (Table 9).

Although the efficiency of conversion of dNTPs into DNA was lower for the dNTP:2NH₄⁺.Mg²⁺ mix at lower concentrations compared to dNTP:4NH₄⁺ decrease in overall efficiency was lower for dNTP:2NH₄⁺.Mg²⁺ over the range of experimental conditions (Table 10).

By using dNTPs with mixed counterions of a monovalent/divalent mixture for Caesium dNTP:2Cs⁺.Mg²⁺ the level of dNTP usage increases from 40 mM with standard dNTP:4Cs⁺ to at least 100 mM and the DNA yield from a peak of 7.65 g/l to 9.40 g/l respectively.

Although the efficiency of conversion of dNTPs into DNA was lower for the dNTP:2Cs⁺.Mg²⁺ mix at lower concentrations compared to dNTP:4Cs⁺ decrease in overall efficiency was lower for dNTP:2Cs⁺.Mg²⁺ over the range of experimental conditions (Table 10).

This experiment was essentially a repeat of Example 1 but using the monovalent cations potassium (K⁺) and caesium (Cs⁺) in place of sodium (Na⁺). Increased concentrations of nucleotides were tested up to 120 mM and reactions monitored over 2 to 6 days by measuring the total DNA produced. The experiment also included the use of ammonium cations (NH₄⁺) repeating experiment 1 but increasing the concentrations of nucleotides to 120 mM.

DNA yields using nucleotide complexes dNTP:2K⁺.Mg²⁺, dNTP:2Cs⁺.Mg²⁺ and dNTP:2NH₄⁺.Mg²⁺ were compared respectively with DNA yields obtained with the pure monovalent cation nucleotide complexes dNTP:4K⁺, dNTP:4Cs⁺ and dNTP:4NH₄⁺ as controls. In the control experiments, an equimolar amount of magnesium (Mg²⁺) to nucleotide was provided in the form of the salt, magnesium chloride.

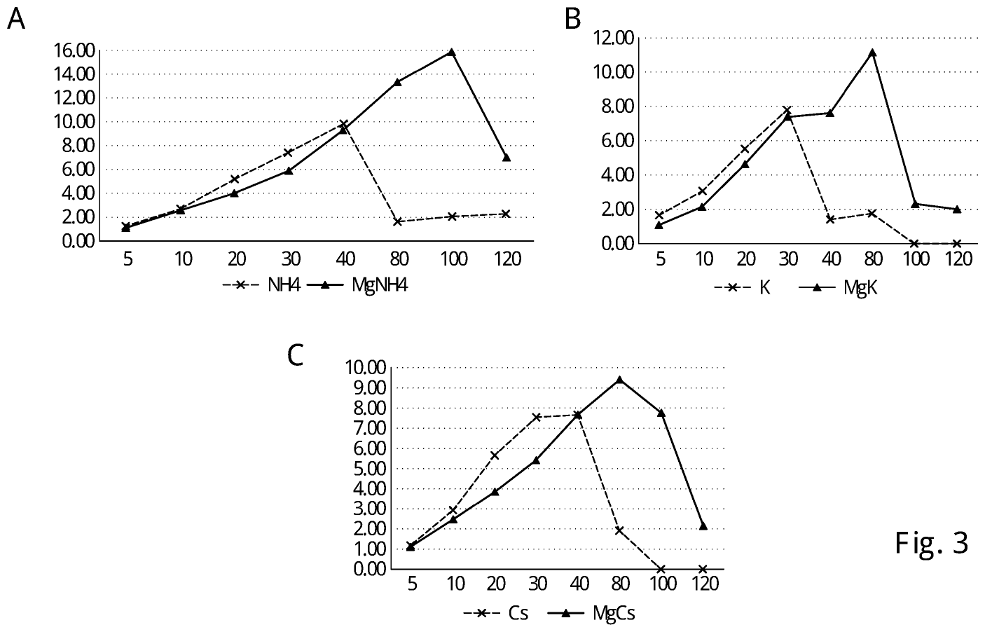

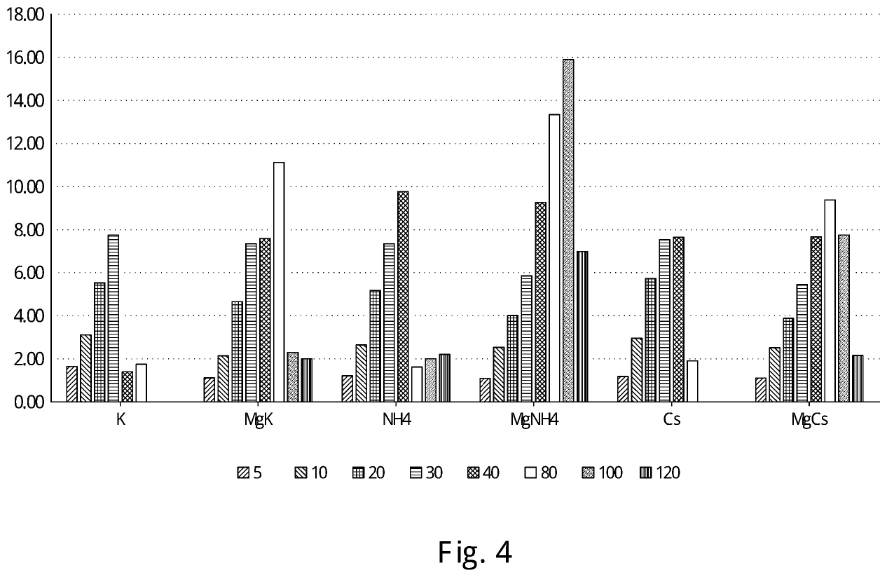

FIGS. 3A, 3B and 3C show graphs of DNA production using the different monovalent cation/divalent cation nucleotide complexes over a period of up to 6 days, with the maximum yield from the data collected plotted. FIG. 4 summarises the results showing the maximum DNA yields at each concentration of nucleotide complex. In all cases, it can be clearly seen that high yields of DNA can only be produced at higher starting nucleotide concentrations if monovalent cation/magnesium nucleotide complexes are used. The greatest effect was observed with the ammonium/magnesium nucleotide complex where 16 g/l DNA was produced from a starting concentration of 100 mM.

Using these complexes compared to the control experiments not only reduces the concentration of monovalent cations in the reaction but also removes the anion on the magnesium salt. Thus, the ionic strength is significantly reduced.

### Example 3

Effect of Different Salts of Magnesium on Magnesium Yields in the Control Experiments

This experiment was carried out to determine whether the nature of the magnesium salt could have a positive or detrimental effect on DNA yield in the control experiments. The magnesium salts compared were magnesium chloride and magnesium acetate since these are widely used in enzymatic DNA synthesis such as PCR.

Experiments were set up as in the previous examples with controls where magnesium was supplied in an equimolar concentration to the nucleotide using magnesium acetate and magnesium chloride. For reference, reactions using nucleotide complexes of sodium (dNTP:2Na⁺.Mg²⁺) and ammonium (dNTP:2NH₄⁺.Mg²⁺) were also conducted.

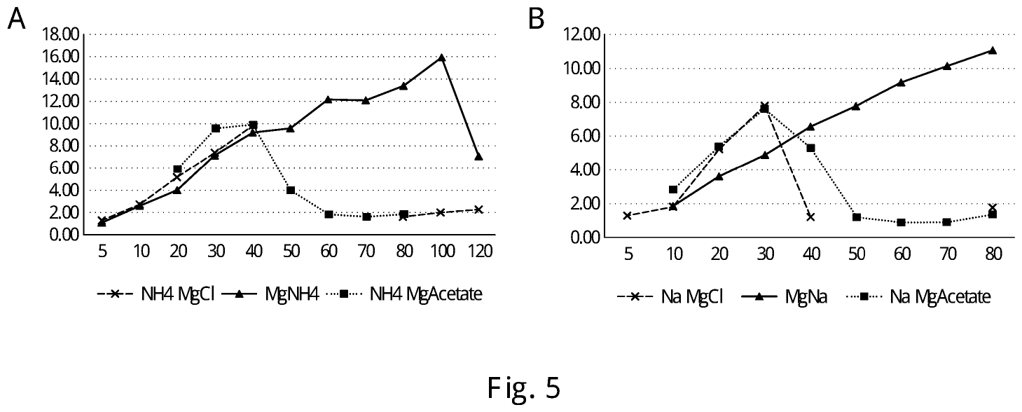

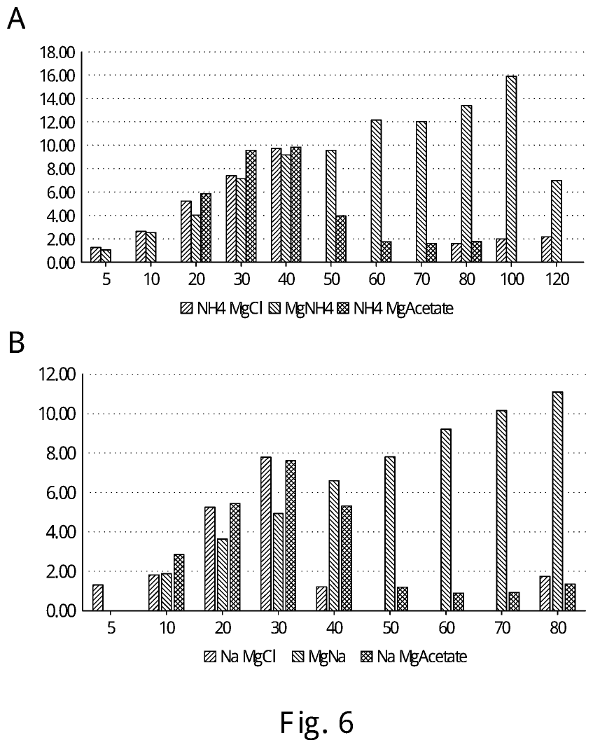

The results in FIGS. 5(A and B) and 6 (A and B) clearly shows that there is no significant effect on DNA yields in the controls when magnesium was supplied either as a chloride or acetate salt.

### Example 4

DNA Amplification Using a dNTP:2NH⁴⁺.Mg²⁺ Nucleotide Complex in the Absence of an External Buffering Agent

DNA Amplification Reaction Setup-Time Course

Reactions were set up at 1000 μl scale as follows: A denaturation mix was prepared, and left at room temperature while the reaction mix was assembled. These were then mixed, and the DNA polymerase and pyrophosphatase added. DNA amplification experiments were then performed at a range of dNTP concentrations with no additional buffering agents added. Reactions were split into 10×100 μl aliquots and incubated at 30° C. and stopped after 24, 48, 72, 96, 120, and 144 hrs by the addition of an Equimolar amount of EDTA to Mg²⁺. To each aliquot 25 μl of both 5M NaCl and 50% PEG 8000 was added, solutions mixed and DNA pelleted by centrifugation in a microcentrifuge (13,000 rpm, 15 minutes) The supernatants were carefully decanted, and the pellets were resuspending in 10000 μl water overnight on an end over end mixer. Reaction DNA concentrations were quantified from UV absorption measurements on a nanodrop spectrophotometer. Data is corrected for the 100× fold increase in reaction volume and concentrations are expressed in g/l of original volume vs dNTP concentrations used.

Reactions were set up as described previously to measure DNA amplification from a range of dNTP:2NH₄⁺.Mg²⁺ nucleotide complex concentrations in the presence and absence of Tris HCl buffer. The control experiments were supplemented with Tris HCl buffer, pH 8.0 to give a final concentration of 30 mM while in the experimental group, Tris buffer was replaced by an equivalent volume of deionised water.

Individual reactions were set up at 100 μl scale and harvested for DNA measurement at daily intervals up to 6 days. Starting concentrations of the dNTP:2NH₄⁺.Mg²⁺ nucleotide complex ranged from 25 mM to 125 mM.

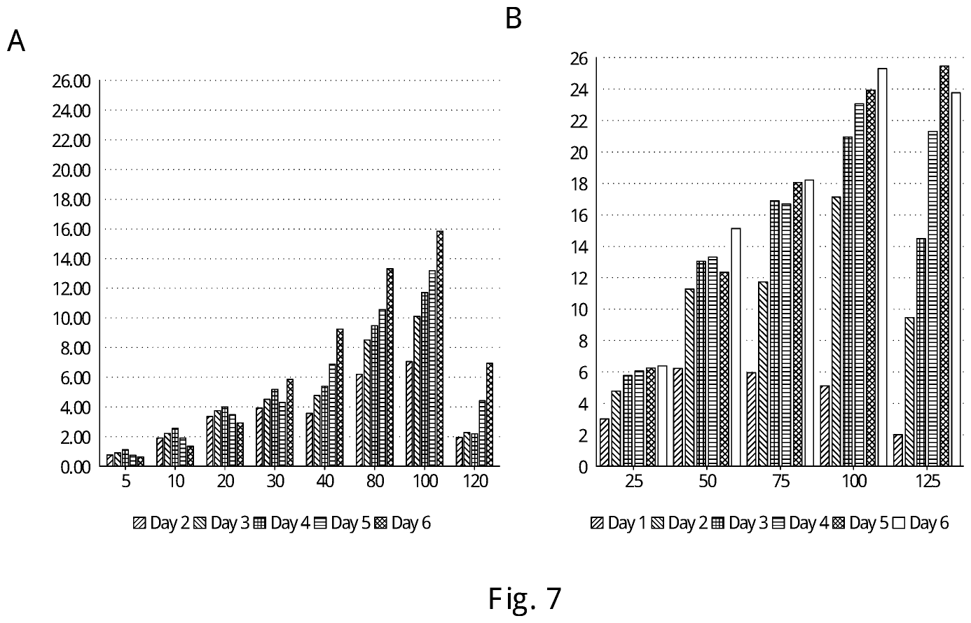

The results in FIGS. 7A and 7B show that there is a highly significant increase in DNA yields of approximately 50% between reactions conducted in the absence of Tris buffer when compared to buffered reactions. This difference is apparent at all comparable concentrations of dNTP:2NH₄⁺.Mg²⁺ nucleotide.

1. A cell-free process for the enzymatic synthesis of DNA in solution comprising obtaining a nucleotide complex, where said nucleotide complex comprises a nucleotide associated with between 0.2 and 2 divalent cations and between 0.2 and 2.5 monovalent cations per nucleotide and adding a nucleotidyltransferase.

2. A cell-free process according to claim 1 wherein the nucleotide complex is a salt.

3. A cell-free process according to any one of claims 1 or 2 wherein said nucleotide complex is electrically neutral.

4. A cell-free process according to claim 3 wherein in order to obtain electrical neutrality the complex associates with one or more hydrogen or hydronium ions.

5. A cell-free process according to any preceding claim wherein the complex is associated with between about 0.5 and 1.5 divalent cations, preferably 1 divalent cation per nucleotide entity.

6. A cell-free process according to any preceding claim wherein the complex is associated with between about 0.2 and 2 monovalent cations.

7. A cell-free process according to any preceding claim wherein said divalent cations are independently selected from: magnesium (Mg²⁺), beryllium (Be²⁺), calcium (Ca²), strontium (Sr²⁺), manganese (Mn²⁺) or zinc (Zn²⁺), preferably Mg²⁺ or Mn²⁺.

8. A cell-free process according to any preceding claim wherein said monovalent cations are independently selected from: an alkali metal, a transition metal, or a polyatomic ion.

9. A cell-free process according to claim 8 wherein said monovalent cations may be independently selected from a polyatomic ion such as an oxonium ion, preferably ammonium or a derivative thereof.

10. A cell-free process according to claim 8 wherein said monovalent cations may be an alkali metal, independently selected from: lithium (Li⁺), sodium (Na⁺), potassium (K⁺), rubidium (Rb⁺), caesium (Cs⁺) or francium (Fr⁺).

11. A cell-free process according to any preceding claim, wherein said nucleotide complex in solution is obtained by mixing nucleotides complexed with divalent cations and a solution of nucleotides complexed with monovalent cations, preferably wherein the divalent and monovalent cations are present at a ratio of less than 4:1 cation:nucleotide, optionally 3.5:1, 3:1 or 2.5:1 or below.

12. A cell-free process according to claim 11 wherein said nucleotide complex with divalent cations is poorly soluble until mixing with nucleotides complexed with monovalent cations.

13. A cell-free process according to claims 11 and 12 wherein the nucleotide complex is soluble.

14. A cell-free process according to any preceding claim wherein said nucleotide complex is obtained at a concentration of at least 30 mM.

15. A cell-free process according to any preceding claim wherein said nucleotide complex is obtained at a concentration of at least 40 mM.

16. A cell-free process according to any preceding claim wherein said nucleotide complex and nucleotidyltransferase form a reaction mixture.

17. A cell-free process according to claim 16 wherein further components are added to the reaction mixture, including but not limited to any one or more of the following:
a) template nucleic acid;
b) primer;
c) primase;
d) denaturing agent, such as sodium or ammonium hydroxide;
e) buffering agents; including buffering salts;
f) pyrophosphatase; and/or
g) magnesium or manganese salts

18. A cell-free process according to claim 17 wherein magnesium or manganese salts are added to the reaction mixture as a co-factor for the nucleotidyltransferase, such that the total ratio of the magnesium and/or manganese to nucleotide does not exceed 2:1.

19. A cell-free process according to any preceding claim wherein said nucleotidyltransferase is a DNA polymerase, preferably a strand-displacing DNA polymerase.

20. A cell-free process according to claim 19 wherein said nucleotidyltransferase is capable of isothermal DNA synthesis.

21. A cell-free process according to any one of claims 1-18 wherein said nucleotidyltransferase does not require a template.
